(19) United States
(12) Patent Application Publication
(10) Pub. No.: US 2005/0239164 A1
(43) Pub. Date: Oct. 27, 2005

(54) Glutathione production

(75) Inventors: Gabriel G. Perrone, Oatley (AU); Ian W. Dawes, Coogee (AU); Christopher M. Grant, Manchester (GB)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Unisearch Limited, Kensington (AU)

(21) Appl. No.: 11/023,709

(22) Filed: Dec. 28, 2004

Related U.S. Application Data
(63) Continuation of application No. PCT/AU03/00837, filed on Jun. 30, 2003.

(30) Foreign Application Priority Data
Jun. 28, 2002 (AU) PS3346

(51) Int. Cl. C12P 21/06; C12N 1/18; C12N 15/74; C07K 5/06

(52) U.S. Cl. 435/69.1; 435/254.2; 435/483; 530/331

(57) ABSTRACT

The present invention relates to a process for the production of glutathione, wherein said process comprises culturing a mutant yeast strain under conditions promoting glutathione production, and wherein said yeast strain has one or more genetic mutations that result in increased secretion of glutathione into the culture medium relative to a parental strain.

### CROSS-REFERENCE TO RELATED APPLICATIONS

This application is a U.S. continuation of PCT Application No. PCT/AU03/000837, filed Jun. 30, 2003, which claims priority to Australian Application No. PS3346, filed Jun. 28, 2002.

### TECHNICAL FIELD

The present invention relates to methods for the production of glutathione by yeasts, as well as yeast mutants for the production of glutathione and for use in bakery applications.

### BACKGROUND ART

Antioxidants are routinely used in foods (including animal feeds) for the protection of, for example, lipids and proteins against oxidative damage, and for avoidance of undesirable reactions such as discoloration and browning. They are also routinely used in the baking industry for control of the rheological properties of dough and the shelf-life of the baked products.

Antioxidants are also now increasingly used in personal health-care products, medications and functional foods (to boost daily dietary intake of antioxidants): oxidation of DNA may directly promote cancer; cardiovascular disease is related to the oxidation of blood lipoproteins which lead to development of atherosclerosis and/or oxidative damage to tissue; and progressive protein oxidation in the eye lens is responsible for the development of cataracts. Studies have shown that increasing intake of oxidants may result in significant reduction of risk of all three of these disease types.

Antioxidants also find use in many other fields such as agriculture, aquaculture, paints, and fermentation media.

Thousands of synthetic and natural antioxidants have been evaluated for the food and pharmaceutical industries, however, synthetic antioxidants are falling into worldwide disfavor due to toxicological problems and consumer reluctance, despite their typically lower cost of production. Even some natural antioxidants are falling into disfavor where these are derived from animal sources (such as cysteine, often included in bread improvers for dough conditioning, and the most viable source of which is bird feathers or human hair). Glutathione, being a natural product, typically derived from non-animal sources, and with known biochemical pathways for utilization within mammalian bodies and having known pathways for removal from mammalian bodies, is an increasingly preferred antioxidant for use in foods, health care products and medications.

The growing or potential markets existing in the pharmaceutical, therapeutic, personal health-care and food/nutritional markets for antioxidants has resulted in increased demand for glutathione and its derivatives.

Although glutathione biosynthesis and degradation have been well studied (FIG. 1 provides a schematic of the glutathione biosynthetic pathway), the genetic mechanisms influencing intra/intercellular glutathione homeostasis have not been fully elucidated.

Commercial production of glutathione has traditionally relied on yeast, in particular selected strains of Saccharomyces or Candida species, and involves growing the yeast for extended periods of up to 5 days. The major proportion of the glutathione produced by the yeast is intracellular but is typically released by heating the harvested concentrated cream yeast (˜18-22% solids) up to 70-80° C. for 10-15 minutes, and during extraction the glutathione would be expected to concentrate to 10-15% of the dry extract solids. The glutathione may then be further fractionated from the extracted solids, typically by chromatographic methods, but the 15% glutathione extracts are typically used without further purification at least in the food industry due to the prohibitive costs that would be associated with further purified product.

These existing methods however suffer the following disadvantages: requirement for significant amounts of heat/energy to extract the glutathione from the yeast; the need to isolate the glutathione from a large amount of other cellular components released from the yeast during the heating process; and the potential contamination of the yeast culture by other organisms (such as lactic acid bacteria, coliforms and wild yeasts) during the lengthy growth period typically used during commercial production.

Therefore, there is a need for an improved process for obtaining glutathione from yeast which can reduce the associated production costs and possibly even result in a cleaner product.

### SUMMARY OF THE INVENTION

The present invention relates to the finding that certain yeast mutants when cultured under appropriate conditions release an increased amount of glutathione into the culture medium than the wild-type, and that this will allow for economic recovery of glutathione from the culture medium without the need to heat the yeast and without the need to remove other components that would typically be released from the yeast during heating.

Further to this, the present invention also relates to novel mutant yeast strains which secrete increased amounts of glutathione into their surrounding culture medium, relative to the wild-type yeast, and the use of these strains for the production of glutathione, including in breadmaking processes and fermentation of beverages.

1. Processes of Producing Glutathione

According to a first embodiment of the invention, there is provided a process for the production of glutathione, wherein said process comprises culturing a mutant yeast strain under conditions promoting glutathione production, and wherein said yeast strain has one or more genetic mutations that result in increased secretion of glutathione into the culture medium relative to the parental strain.

The glutathione secreted into the culture medium can, optionally, be isolated from the culture medium by techniques well known to those of skill in the art. It has been surprisingly found that yeast mutants unable to synthesize or which have a reduced ability to synthesize certain metabolites and/or essential growth factors, such as amino acids or their precursors, secrete increased amounts of glutathione into the surrounding culture medium.

Thus, according to one aspect of the process of the invention, the yeast strain is incapable of the synthesis of one or more metabolites and/or essential growth factors which are included in the culture medium in limiting amounts.

According to another aspect of the process of the invention, the yeast strain has a mutation that reduces the ability of the strain to synthesize one or more proteins, metabolites and/or essential growth factors which may optionally be included in the culture medium in limiting amounts, depending on the capacity of the yeast strain to synthesize said proteins, metabolites and/or essential growth factors.

Typically the metabolite(s) and/or essential growth factor(s) for which the yeast is deficient, or for which it has a reduced ability for synthesis, is an amino acid or a precursor or metabolite thereof.

Even more typically, the metabolite(s) and/or essential growth factor(s) for which the yeast is deficient, or for which it has a reduced ability for synthesis, is leucine, isoleucine and/or valine, or precursors or metabolites thereof, and more typically is leucine or precursors or metabolites thereof.

It has also been found that a mutation in any one of a number of cellular processes in yeast may lead to increased secretion of glutathione by the yeast into the surrounding culture medium.

Thus, according to another aspect of the invention, the yeast strain has a mutation selected from the following groupings, which may overlap:
i) mutation in a gene or genes encoding components of the mitochondrial respiratory chain or nuclear genes encoding proteins that maintain the integrity of the mitochondrial genome or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26 S proteosome;
viii) mutation in a gene or genes affecting transportation of glutathione across the yeast cell membrane;
ix) mutation in a gene or genes affecting glutathione degradation; and
x) mutation in a gene or genes involved in vacuolar function.

The yeast strain may have more than one mutation within any one or more of the above groups (i) to (x).

Yeast strains which could be used in the process of the present invention may include yeast selected from the genera Saccharomyces, Candida, Kluyveromyces, Pichia, Rhodotorula, Hansenula, Debaryomyces, Torulopsis or the fission yeast genus Schizosaccharomyces. However, according to a preferred aspect of the methods of the invention, the yeast strain is a Saccharomyces species, and more preferably a strain of Saccharomyces cerevisiae.

According to a preferred aspect of the process according to the invention, the yeast strain has mutations in two or more of gene groups (i) to (x) listed above. Even more preferably, such a yeast strain will also be a mutant for the synthesis of one or more proteins, metabolites and/or essential growth factors, wherein the mutant is unable to synthesize said one or more proteins, metabolites and/or essential growth factors or has a restricted ability for synthesis of said one or more metabolites and/or essential growth factors. Typically the one or more metabolites and/or essential growth factors are amino acids or precursors or metabolites thereof. More typically the one or more metabolites and/or essential growth factors are selected from leucine, isoleucine or valine or precursors or metabolites thereof, and even more typically from leucine or precursors or metabolites thereof.

According to another aspect of the invention, the yeast strains for use in the process according to the invention may have at least one mutation selected from groups (i) to (x) as described above, in addition to genetic manipulation resulting in overexpression of the glutathione synthesis pathway. Such manipulations resulting in, for example, overexpression of gammaglutamylcysteine synthetase (GSH1), glutathione synthetase (GSH2) or GSH1 and GSH2.

According to another preferred aspect of the process of the invention, the conditions under which the yeast strain is cultured include maintaining the yeast in aerobic growth which provides for increased glutathione production and secretion.

According to another preferred aspect of the process of the invention, the conditions under which the yeast strain is cultured include reduced pH, typically a pH of less than about 6, which has also been found to result in increased glutathione production and secretion. Typically the pH of the culture medium is between about 2.5 and 5, more advantageously between about 3 and 4.5, even more advantageously between about 3 and 4, and even more preferably about 3.5.

According to another preferred aspect of the process of the invention, the conditions under which the yeast strain is cultured include the presence of monovalent cations, which has also been found to result in increased glutathione production and secretion. Typically, the monovalent cations are selected from sodium, potassium, rubidium and caesium, preferably sodium or potassium and even more preferably potassium. The monovalent cation is typically provided as a salt, preferably as the chloride, and the concentration of the salt in the culture medium is typically from about 50 mM to 500 mM, more typically about 50 to 350 mM, more typically from about 100 to 250 mM, even more typically from about 100 to 200 mM, and preferably about 150 mM.

According to a second embodiment of the invention, there is provided a process for the production of glutathione comprising culturing a yeast strain under conditions promoting glutathione production, wherein the culture medium comprises myo-inositol.

Typically the resulting glutathione is isolated from the culture medium.

According to a preferred aspect of this embodiment, the process is a process according to the invention utilizing a mutant yeast strain as described above.

Typically, where myo-inositol is included in the culture medium in a process of the invention, the concentration of myo-inositol is from about 0.01 mM to 100 mM (1.8 mg/L to 18000 mg/L), more typically about 0.1 to 10 mM, more typically from about 0.2 to 5 mM, even more typically from about 0.5 to 2 mM, and more typically about 1 mM.

According to a preferred aspect of the processes of the invention, the culture medium comprises myo-inositol and elevated levels of a carbon source.

Typically the carbon source used in processes of the invention is selected from fermentable sugars, more typically glucose or fructose or a combination thereof, and/or from oligosaccharides which are homo- or hetero-oligomers comprising fermentable sugar moieties, such as sucrose or maltose, even more typically sucrose.

Alternatively, the carbon source may be a non-fermentable carbon source, more typically ethanol, glycerol, lactate, galactose or raffinose.

Typically, a carbon source is included in a culture medium at a concentration of about 1-2% w/v. Where elevated carbon source levels are to be included in the culture medium in combination with myo-inositol, the concentration of the carbon source in the initial, uninoculated, culture medium, is typically greater than about 2% w/v, more typically between about 2% and 10% w/v, more typically between about 3% and 8% w/v; more typically between about 3% and 6% w/v, and even more typically about 4% w/v.

According to a preferred aspect of a process of the invention, the process comprises growth of the yeast strain by batch-wise culture. If desired, the glutathione may then be extracted from the culture medium by any of a number of known methods, such as chromatographic methods.

Alternatively, a process of the invention comprises growth of the yeast by continuous culture, allowing for continuous harvesting of culture medium and therefore recovery of secreted glutathione.

According to yet another aspect of a process of the invention, the process comprises dough preparation. Doughs prepared by this process, or baked products derived therefrom are also provided.

According to yet another aspect of a process of the invention, the process comprises preparation of a fermented product. Fermented products prepared by said process are also provided.

2. Yeast Strains for Glutathione Production and/or Baking or Fermentation Applications

The invention also relates to novel strains obtained by any form of directed mutagenesis, consisting of generating, preferably in industrial strains of yeasts, particularly baker's yeast, or in the starting haploids that served for construction of the industrial strains, mutations, monogenic or not, giving the required phenotype in the strains. This includes strains selected after conventional mutation treatment, for example using chemical/physical agents or molecular biological techniques or standard selection recombination methods to generate multiple mutants.

Thus, according to a third embodiment of the invention, there is provided a mutant yeast strain having at least two mutations selected from the following groupings, which may overlap:
i) mutation in a gene or genes encoding components of the mitochondrial respiratory chain or nuclear genes encoding proteins that maintain the integrity of the mitochondrial genome or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26 S proteosome;
viii) mutation in a gene or genes affecting transportation of glutathione across the yeast cell membrane;
ix) mutation in a gene or genes affecting glutathione degradation;
x) mutation in a gene or genes involved in vacuolar function.

The yeast strain may have more than one mutation within one of the above groups (i) to (x).

Yeast strains which are contemplated by the present invention include, but are not necessarily limited to yeast selected from the genera Saccharomyces, Candida, Kluyveromyces, Pichia, Rhodotorula, Hansenula, Debaryomyces, Torulopsis or the fission yeast genus Schizosaccharomyces. However, according to a preferred aspect of this embodiment of the invention, the yeast strain is a Saccharomyces species, and more preferably a strain of Saccharomyces cerevisiae.

According to a preferred aspect of this embodiment of the invention, the yeast strain has mutations in one or more of mutation groups (i) to (x) listed above and will also be a mutant for the synthesis of one or more proteins, metabolites and/or essential growth factors, wherein said mutant is unable to synthesize said one or more proteins, metabolites and/or essential growth factors or has a restricted ability to synthesize said one or more proteins, metabolites and/or essential growth factors. Typically the one or more metabolites and/or essential growth factors are amino acids or precursors or metabolites thereof. More typically the one or more metabolites and/or essential growth factors are selected from leucine, isoleucine or valine or precursors or metabolites thereof, and even more typically is leucine or precursors or metabolites thereof.

According to a preferred aspect of this embodiment of the invention, the yeast strain may have at least one mutation selected from groups (i) to (x) as described above, in addition to genetic manipulation resulting in overexpression of the glutathione synthesis pathway. Such manipulations resulting in, for example, overexpression of gammaglutamylcysteine synthetase (GSH1), glutathione synthetase (GSH2) or GSH1 and GSH2.

According to a fourth embodiment of the invention, there is provided a yeast strain herein described as BSO4ycf1. The BSO4 mutation has been identified as a defect in the HAC1 gene (YFL031W).

According to a fifth embodiment of the invention, there is provided a method of preparing a dough comprising combining a yeast strain according to the invention with other dough components. Doughs prepared by this method, and baked products derived therefrom, are also provided.

According to a sixth embodiment of the invention, there is provided a method of producing a fermented product comprising adding to the unfermented precursor component(s) of said product a yeast strain according to the invention. Fermented products obtained by this method are also provided.

3. Compositions Comprising Glutathione Obtained by the Process of the Invention, and Uses Thereof.

According to a seventh embodiment of the invention, there is provided glutathione obtained by a process of the invention. The glutathione may be provided as a concentrated form of the culture medium or it may be purified to any desired degree.

The glutathione may be used in a wide variety of applications including, but not restricted to personal health care, pharmaceuticals, nutraceuticals, cosmetics, food (including bakery and fermentation technology) and animal feeds, agriculture, aquaculture, paints, and fermentation media. For pharmaceutical applications the glutathione is preferably provided as a purified compound, typically greater than 60% pure, more typically greater than 70% pure, more typically greater than 80% pure, even more typically greater than 90% pure, and more preferably greater than 95% pure.

According to an eighth embodiment of the invention, there is provided a personal health care composition comprising glutathione obtained by a process of the invention and a pharmaceutically or topically acceptable carrier.

According to a ninth embodiment of the invention, there is provided a pharmaceutical composition comprising glutathione obtained by a process of the invention and a pharmaceutically acceptable carrier.

According to a tenth embodiment of the invention, there is provided a food or nutraceutical composition comprising glutathione obtained by a process of the invention in combination with one or more food components. The food/nutraceutical composition may be selected from liquids, semi-solids and solids.

According to an eleventh embodiment of the invention, there is provided a dough or bread improving composition comprising glutathione obtained by a process of the invention and a suitable carrier. The carrier may be selected from a wide variety of bakery acceptable ingredients, including flour and/or sugar and the composition may also include other bread improving ingredients such as enzymes (including cellulases, glucanases, amylases, xylanases, arabinoxylanases, dextrinases, maltases, etc.). The composition may be in the form of a powder, granulate or liquid.

According to a twelfth embodiment of the invention, there is provided an animal feed additive comprising glutathione obtained by a process of the invention and a suitable carrier. The carrier may be selected from a wide variety of acceptable animal feed ingredients, such as flour (including wheat, corn or soy), and the composition may also include other animal feed additives including those which improve the digestibility of the food such as enzymes (including cellulases, glucanases, amylases, xylanases, arabinoxylanases, dextrinases, maltases, etc.). The composition may be in the form of a powder, granulate or liquid.

According to a thirteenth embodiment of the invention, there is provided an animal health care composition comprising glutathione obtained by a process of the invention and a veterinary acceptable carrier.

According to a fourteenth embodiment of the invention, there is provided a method for preventing oxidative damage in the circulation or tissues of a mammal, said method comprising administering to said mammal an effective amount of a composition comprising glutathione obtained by a process of the invention.

According to a fifteenth embodiment of the invention, there is provided a method of protecting a food product from oxidative deterioration comprising adding to said food product an effective amount of glutathione obtained by a process of the invention or a composition comprising it. Food products prepared by said method are also provided. The food product may be liquid, semi-solid or solid.

According to a sixteenth embodiment of the invention, there is provided a method of preparing a dough comprising combining dough components with an effective amount of glutathione obtained by a process of the invention. Doughs prepared by this method, or baked products derived therefrom are also provided.

### BRIEF DESCRIPTION OF THE DRAWINGS

FIG. 1 shows a representation of the biosynthetic pathway for glutathione in yeast.

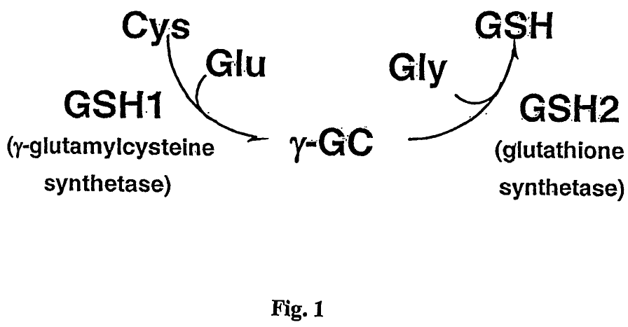

FIG. 2 shows intracellular and extracellular glutathione production with time after inoculation into fresh medium for a mutant strain as compared to the parental strain.

FIG. 3 is a graph illustrating glutathione production (intracellular and extracellular) with time after inoculation into fresh medium for a deletion mutant (Δvps27) of yeast strain BY4743 (Winzeler E. A. et al., (1999), Science 285: 901-906) as compared to the parental strain.

FIG. 4 is a bar chart showing increased glutathione secretion by the dominant mutant RAS2Val19 as compared to ras2 and the parental strain.

FIG. 5 illustrates potential interactions between cellular compartments/components, associated genes/mutations and glutathione secretion (relative to the parental strain—values in brackets represent the ratio of glutathione secreted by the mutant to that secreted by the parental strain).

FIG. 6 illustrates potential interactions between mitochondrial respiratory chain components, associated genes/mutations and glutathione secretion (relative to the parental strain—values in brackets represent the ratio of glutathione secreted by the mutant to that secreted by the parental strain).

FIG. 7 is a graph illustrating extracellular glutathione vs pH for a mutant yeast strain as compared to the parental strain.

FIG. 8 is a bar chart of extracellular glutathione vs pH for a deletion mutant as compared to the parental strain.

FIG. 9 shows two bar charts—one for extracellular glutathione and the other for corresponding intracellular glutathione produced at pH 3.5 or 6.0 for deletion mutants of yeast strain BY4743 as compared to the parental strain.

FIG. 10 provides two bar charts illustrating comparative glutathione productions, both intracellular and extracellular for a mutant in the presence of different monovalent salts as compared to the parental strain.

FIG. 11 is a bar chart showing increased extracellular glutathione levels produced by a wild-type haploid strain grown on SD medium, SD medium supplemented with 200 mg/L myo-inositol, SD medium supplemented with 4% w/v glucose, and SD medium supplemented with 200 mg/L myo-inositol and 4% w/v glucose.

FIG. 12 is a bar chart illustrating extracellular glutathione for two yeast single mutants and a double mutant relative to the parental strain.

FIG. 13 is a bar chart showing extracellular glutathione levels produced by a mutant yeast strain having the combined deletion of HGT1 and loss of mitochondrial respiratory function (petite cells).

FIG. 14 is a bar chart showing extracellular glutathione levels produced by wild-type haploid strains (CY4 and BY4742), single mutants thereof, and diploids obtained by mating the haploids.

### DEFINITIONS

The term “Yeast” encompasses any group of unicellular fungi that reproduce asexually—by budding or fission—and sexually—by the production of ascospores. Yeast cells may occur singly or in short chains, and some species produce a mycelium. Typically the yeast will be a member of the genera Saccharomyces, Candida, Kluyveromyces, Pichia, Rhodotorula, Hansenula, Debaryomyces, Torulopsis or the fission yeast genus Schizosaccharomyces. However, typically, the yeast is a Saccharomyces species, more typically a strain of Saccharomyces cerevisiae, and even more typically an industrial baker's yeast strain.

“Increased secretion of glutathione into the culture medium relative to the wild-type” as referred to herein means secretion of at least 50% more, preferably at least 100% more glutathione by the mutant, relative to the parental strain when grown as described in Example 1 herein. The glutathione secretion by the mutant relative to the wild-type may be expected to vary depending on the growth conditions.

The term “mutation” encompasses any mutation which results in a “functional” deficiency, irrespective of how the genes have been mutated. Mutations may typically include deletion mutations, point mutations, insertion or substitution mutations, frame-shift mutations or any other method that results in inactivation of a gene (including RNAi approaches to selectively inactivating gene expression). The terms “mutant yeast”, “mutant strain” and “mutant yeast strain” as used herein have corresponding meanings.

As used herein, the term “aerobic growth” refers to the growth phase in which yeast is grown in the presence of oxygen. In batch growth of yeast in culture flasks on a given amount of fermentable sugar, aerobic growth on ethanol occurs after the ‘diauxic shift’ when all the fermentable sugars have been consumed, consumption of sugars to produce ethanol stops and the yeast's physiology alters to adapt to growth on ethanol by respiration. In commercial scale fermenters, yeast is typically grown with exponential sugar feeding rates, after the yeast has started to efficiently consume the ethanol —although ethanol is also produced during such an ‘aerobic’ yeast fermentation, this is generally consumed at a greater rate than it is produced and this growth pattern is also encompassed within the term ‘aerobic growth’ as used herein.

The term “isolated”, where used in relation to glutathione, indicates that the material in question has been removed from a cell culture, and associated impurities either reduced or eliminated. Essentially, the ‘isolated’ material is enriched with respect to other materials extracted from the same source (i.e., on a molar basis it is more abundant than any other of the individual species extracted from a given source), and preferably a substantially purified fraction is a composition wherein the ‘isolated’ material comprises at least about 60 percent (on a molar basis) of all molecular species present. Generally, a substantially pure composition of the material will comprise more than about 80 to 90 percent of the total of molecular species present in the composition. Most preferably, the ‘isolated’ material is purified to essential homogeneity (contaminant species cannot be detected in appreciable amounts).

An “effective amount”, as referred to herein, includes a sufficient, but non-toxic amount of substance to provide the desired effect. The “effective amount” will vary from application to application (such as from dough preparation to use in pharmaceutical compositions) and even within applications (such as from subject to subject in pharmaceutical applications, and from dough to dough in baking applications). For any given case, an appropriate “effective amount” may be determined by one of ordinary skill in the art using only routine experimentation.

The term “carbon source”, as referred to herein, includes carbohydrates which can be taken up by yeast cells and converted to energy through fermentative and/or aerobic growth pathways. Typically, the carbon source is a fermentable sugar, typically glucose or fructose or a combination thereof, and/or from oligosaccharides which are homo- or hetero-oligomers comprising fermentable sugar moieties, such as sucrose or maltose, even more typically sucrose. Typically the carbon source is selected from glucose, fructose and/or sucrose (which in commercial sugar sources such as molasses typically occur together), although these are initially utilized through the fermentative pathway to produce primarily ethanol, which is then utilized through the oxidative pathway.

In the context of this specification, the term “comprising” means “including principally, but not necessarily solely”. Variations of the word “comprising”, such as “comprise” and “comprises”, have correspondingly varied meanings.

### BEST MODE OF PERFORMING THE INVENTION

1. Processes for the Production of Glutathione

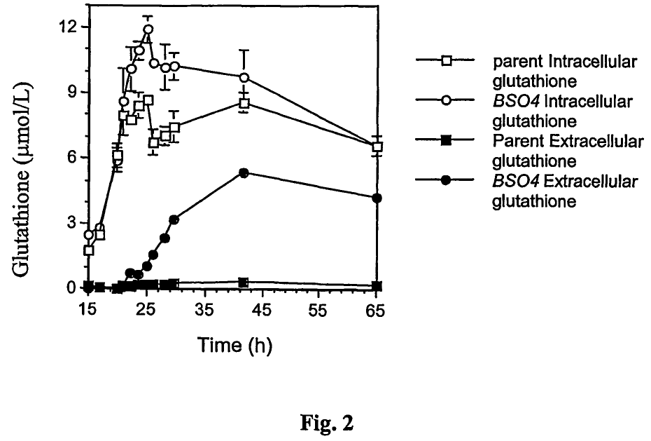

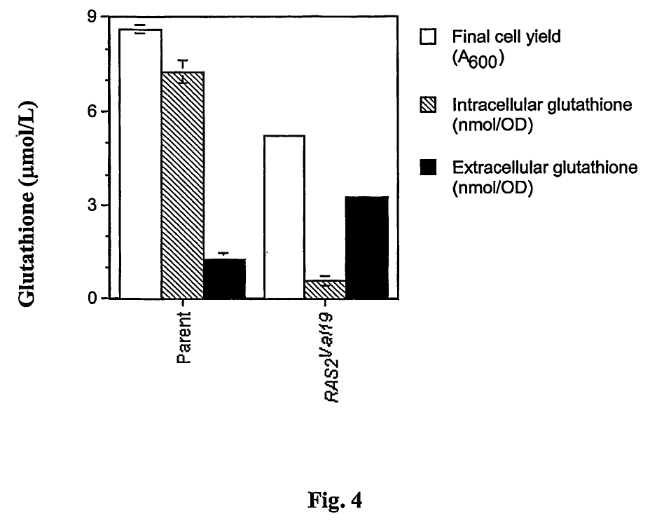

The present invention relates to a finding that certain types of mutation in yeasts can result in significantly increased secretion of glutathione relative to a parental strain (examples provided in FIGS. 2 to 4), which typically secrete only a small fraction of the glutathione produced. Thus the present invention relates to a process for the production of glutathione, wherein said process comprises culturing a mutant yeast strain under conditions promoting glutathione production, and optionally isolating glutathione from the culture medium, and wherein said yeast strain has one or more genetic mutations that result in increased secretion of glutathione into the culture medium relative to a parental strain.

It has been surprisingly found that yeast mutants unable to synthesize or which have a reduced ability to synthesize certain amino acids secrete increased amounts of glutathione into the surrounding culture medium. This increased secretion, relative to a parental strain, can be reduced if not eliminated by supplementing the yeast with a compensating amount of the required amino acid or by transforming the strain back to a leucine-synthesizing phenotype.

According to one aspect, the yeast strain is incapable of the synthesis of one or more metabolites and/or essential growth factors which are included in the culture medium in limiting amounts.

According to another aspect, the yeast strain has a mutation that reduces the ability of the strain to synthesize one or more proteins, metabolites and/or essential growth factors which may optionally be included in the culture medium in limiting amounts, depending on the capacity of the yeast strain to synthesize said proteins, metabolites and/or essential growth factors.

Typically the metabolite(s) and/or essential growth factor(s) for which the yeast is deficient, or for which it has a reduced ability for synthesis, is an amino acid or a precursor or metabolite thereof.

Even more typically, the metabolite(s) and/or essential growth factor(s) for which the yeast is deficient, or for which it has a reduced ability for synthesis, is leucine, isoleucine and/or valine or precursors or metabolites thereof, and more typically is leucine or precursors or metabolites thereof.

Typically the metabolite which the strain is unable to synthesize, or which it has a reduced ability for the synthesis of, is included in the growth medium at sub-optimal levels, typically approximately half-optimal levels.

It has also been found that a mutation in a number of pathways in yeast may lead to increased secretion of glutathione by the yeast into the surrounding culture medium.

Thus, according to another aspect, the yeast strain has a mutation in one or more of the following groupings, which may overlap:
i) mutation in a gene or genes encoding components of the mitochondrial respiratory chain or nuclear genes encoding proteins that maintain the integrity of the mitochondrial genome or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26S proteosome;
viii) mutation in a gene or genes affecting transportation of glutathione across the yeast cell membrane;
ix) mutation in a gene or genes affecting glutathione degradation; and
x) mutation in a gene or genes involved in vacuolar function.

The yeast strain may also have more than one mutation within one of the above groups (i) to (x).

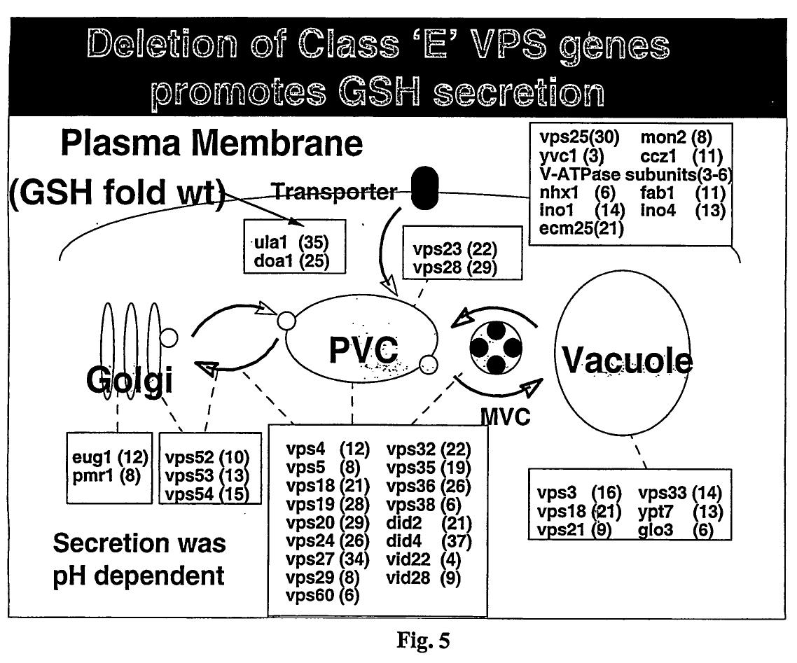

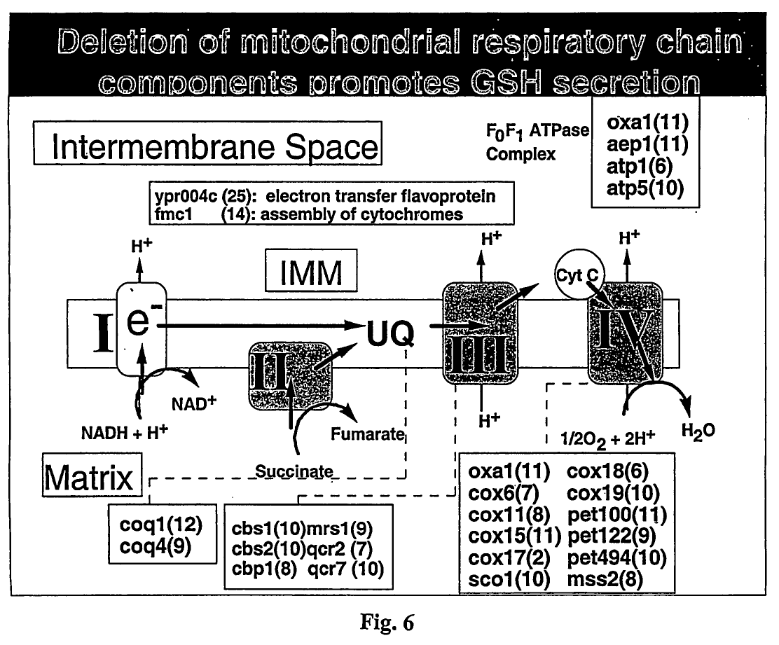

FIGS. 5 and 6 illustrate potential ways in which some of the above listed mutation types may affect the secretion of glutathione from yeast cells.

Yeast strains which could be used in a process of the present invention may include yeast selected from the genera Saccharomyces, Candida, Kluyveromyces, Pichia, Rhodotorula, Hansenula, Debaryomyces, Torulopsis or the fission yeast genus Schizosaccharomyces. However, according to a preferred aspect of the methods of the invention, the yeast strain is a Saccharomyces species, more preferably a strain of Saccharomyces cerevisiae and even more preferably an industrial strain of baker's yeast which can better withstand the conditions to which yeast are exposed during industrial-scale fermentations.

According to another aspect, the yeast is a mutant strain of Saccharomyces cerevisiae which has at least a mutation in a gene encoding a component of the mitochondrial respiratory chain or nuclear genes encoding proteins that maintain the integrity of the mitochondrial genome, wherein said gene is selected from the following: YBL009W(ATP1); YBR003W(COX1); YBR037C(SCO1); YBR191W(RPL21α); YBR220C; YBR268W(MRPL37); YCR046C(IMG1); YDL069C (CBS1); YDL107W (MSS2); YDL202W(MRPL11); YDR079W(PET100); YDR175C(RSM24); YDR197W (CBS2); YDR204W(COQ4); YDR298C(ATP5); YDR322W(MRPL35); YDR337W (MRPS28); YDR462W(MRPL28); YDR529C(QCR7); YER017C(AFG3); YER141W (COX15); YER153C(PET122); YER154W(OXA1); YFL034W(MRPL7); YGR062C (COX18); YGR171C (MSM1); YGR220C (MRPL9); YGR257C; YHL004W(MRP4); YHL038C (CBP2); YHR011W(DIA4); YHR051 W (COX6); YHR120w (MSH1); YHR147C (MRPL6); YIL006W; YIL018W(RPL2B); YIL065C (FIS1); YIL070C (MAM33); YIL093C(RSM25); YIL098C (FMC1); YIR021 W(MRS1); YJL063C (MRPL8); YJL102W(MEF2); YJL166W(QCR8); YJL209W(CBP1); YJR144W (MGM101); YKL003C (MRP17); YKL032C (IXR1); YKR006C (MRPL13); YLL009C (COX17); YLL018C-A (COX19); YLR067C(PET309); YLR139C(SLS1); YLR295C (HSP60); YLR369W (SSQ1); YML078W (CPR3); YMR064 W (AEP1); YMR072 W (ABF2); YMR150C(IMP1); YMR193W(MRPL24); YMR228W(MTF1); YNL177C; YNR036C; YNR037C(RSM19); YNR045W(PET494); YOL009C(MDM12); YOL033W (MSE1); YOL095C (HMI1); YOR026W(BUB3); YPL132W(COX11); YPL183W-A; YPR004C; YPR166C (MRP2); and YPR191W(QCR2).

According to another aspect, the yeast is a mutant strain of Saccharomyces cerevisiae which has at least a mutation in a gene affecting the levels of NADH and NAD⁺, wherein said gene is selected from genes encoding enzymes which catalyze the synthesis of glycerol, ethanol and/or genes the expression of which suppress or result in competition for the GLN1, GLT1 glutamate synthesis pathway. Typically these genes are selected from YIL053 W (RHR2), YOR375C (GDH1) and YNL229c (URE2).

According to another aspect, the yeast is a mutant strain of Saccharomyces cerevisiae which has at least a mutation in a gene affecting the assimilation and metabolism of nitrogen in the cell, wherein said gene is selected from: YDR300C (PRO1); YDR448W(ADA2); YEL009C (GCN4); YEL062W(NPR2); YGL227W (VID30); YGR252W(GCN5); YNL106C (INP52); YNL229C (URE2); YOR375C (GDH1); and YPL254W(HFI1).

According to another aspect, the yeast is a mutant strain of Saccharomyces cerevisiae which has at least a mutation in a gene encoding regulatory components of the Ras/cAMP/PKA pathway or otherwise affecting the activity of the Ras/cAMP/PKA pathway, and wherein said gene is selected from YOL081 W(IRA2); YOR360C (PDE2); and YNL098C (RAS2; RAS2Val19 dominant mutation).

According to another aspect, the yeast is a mutant strain of Saccharomyces cerevisiae which has at least a mutation in a gene affecting endosomal function, wherein said gene is selected from: YCL008C (VPS23; STP22); YDR456W(NHX1); YJR102C (VPS25); YKL002 W (DID4); YKL041 W (VPS24); YKR035 W-A (DID2); YLR025 W (VPS32/SNF7); YLR119W(SRN21VPS37); YLR417W(VPS36); YMR077C(VPS20); YNR006W(VPS27); YPL065W(VPS28); and YPR173C(VPS4).

According to another aspect, the yeast is a mutant strain of Saccharomyces cerevisiae which has at least a mutation in a gene affecting endoplasmic reticulum function, the Golgi to endosome to vacuole transportation pathway, or vacuolar function wherein said gene is selected from: YFL031W(HAC1), YDR027C(LUV1/VPS54); YDR323C(PEP7/VPS19); YDR484W(VPS52/SAC2); YBR131W(CCZ1); YDR486C (VPS60); YHR012W(VPS29); YJL154C (VPS35); YLR148W(VAC1/PEP3/VPS18); YML001W(YPT7); and YOR036W(PEP12/VPS6).

According to another aspect, the yeast is a mutant strain of Saccharomyces cerevisiae which has at least a mutation in a gene affecting ubiquitin levels and ubiquitin-mediated proteolysis via the 26S proteosome, wherein said gene is selected from: YBR173C(UMP1); YER151C (UBP3); YFR010W(UBP6); YHL011C(PRS3); YKL213C(DOA1); YNR051C(BRE5); YPL003W(ULA1); and YPL074W(YTA6).

According to another aspect, the yeast is a mutant strain of Saccharomyces cerevisiae which has at least a mutation in a gene involved in transportation of glutathione across the yeast cell membrane, wherein said gene is YDR135C (YCF1) or YJL212C (HGT1).

According to a preferred aspect, the yeast strain has mutations in two or more of groups (i) to (x) listed above. Examples of such mutations are described in paragraph 2.1 below.

Even more preferably, such a yeast strain will also be a mutant for the synthesis of one or more proteins, metabolites and/or essential growth factors, wherein the mutant is unable to synthesize said one or more proteins, metabolites and/or essential growth factors or has a restricted ability for synthesis of said one or more metabolites and/or essential growth factors. Typically the one or more metabolites and/or essential growth factors are amino acids or precursors or metabolites thereof. More typically the one or more metabolites and/or essential growth factors are selected from leucine, isoleucine or valines or precursors or metabolites thereof, and even more typically from leucine or precursors or metabolites thereof.

According to another aspect, the yeast strains for use in a process according to the invention may have at least one mutation selected from groups (i) to (x) as described above, in addition to genetic manipulation resulting in overexpression of the glutathione synthesis pathway. Such manipulations resulting in, for example, overexpression of gammaglutamylcysteine synthetase (GSH1), glutathione synthetase (GSH2) or GSH1 and GSH2.

According to another preferred aspect, the conditions under which the yeast strain is cultured include maintaining the yeast in aerobic growth which provides for increased glutathione production and secretion.

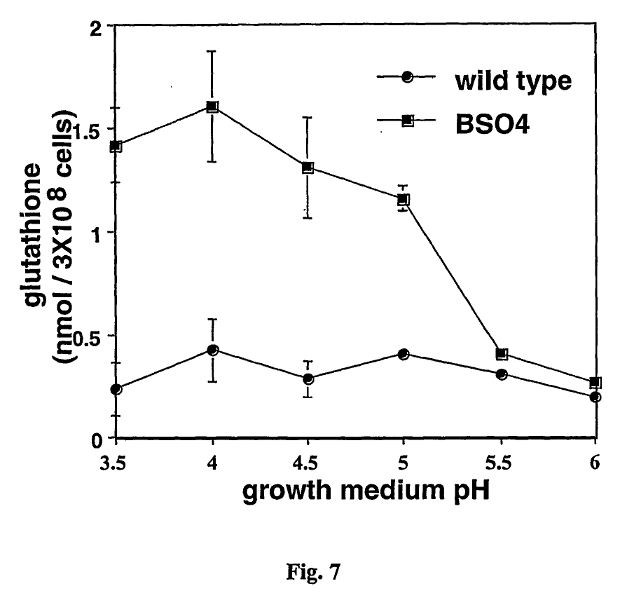

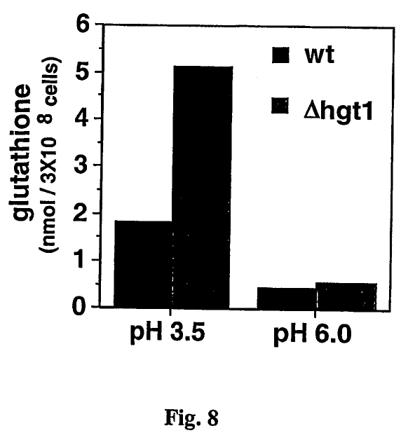

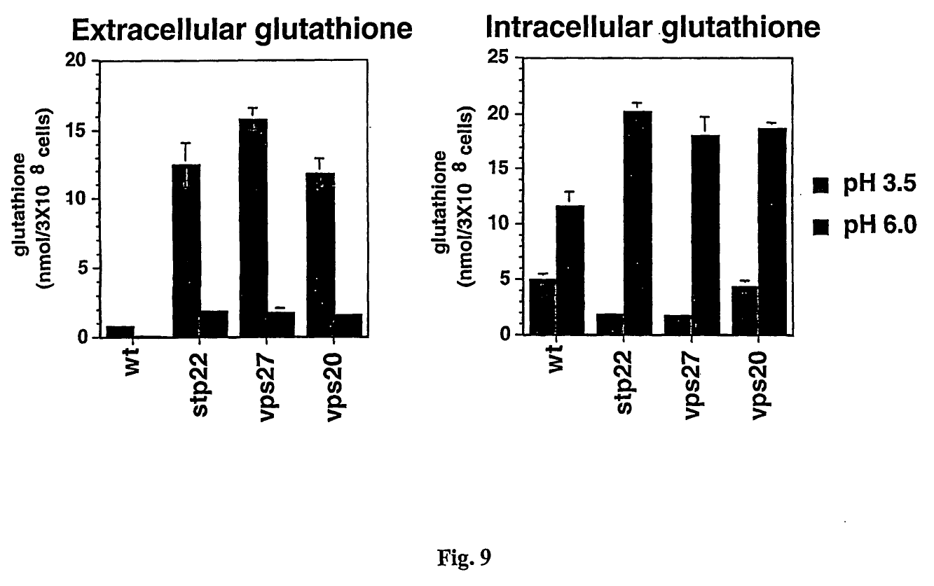

According to another preferred aspect, the conditions under which the yeast strain is cultured include reduced pH, typically a pH of less than about 6, which has also been found to result in increased glutathione production and secretion. Typically the pH of the culture medium is between about 2.5 and 5, more advantageously between about 3 and 4.5, even more advantageously between about 3 and 4, and even more preferably about 3.5. FIGS. 7 to 9 illustrate results of extracellular glutathione levels produced by representative strains at either pH 3.5 or pH 6.0 or intermediate values (the culture conditions being as described in Example 3).

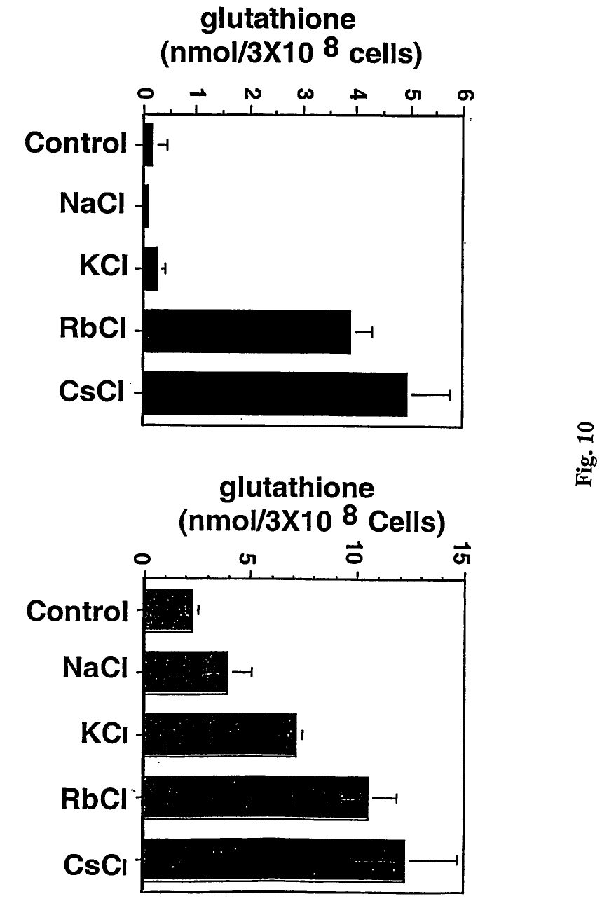

According to another preferred aspect, the conditions under which the yeast strain is cultured include the presence of monovalent cations, which has also been found to result in increased glutathione production and secretion. Typically, the monovalent cations are selected from sodium, potassium, rubidium and caesium, preferably sodium or potassium and even more preferably potassium. The monovalent cation is typically provided as a salt, preferably as the chloride, and the concentration of the salt in the culture medium is typically from about 50 mM to 500 mM, more typically 50 to 350 mM, more typically from 100 to 250 mM, even more typically from 100 to 200 mM, and preferably about 150 mM. Mutations that are likely to affect the natural metabolism/homeostasis of these cations are also expected to play a role in glutathione homeostasis and are contemplated by the present invention. FIG. 10 illustrates extracellular glutathione levels produced by the mutant strain BSO4 and the wild-type when grown without, or in the presence of NaCl, KCl, RbCl or CsCL (the culture conditions being as described in Example 3).

The addition of myo-inositol to the culture medium has also been found to result in increased glutathione production and secretion by yeast strains.

Thus, according to a second embodiment of the invention, there is provided a process for the production of glutathione comprising culturing a yeast strain under conditions promoting glutathione production, wherein the culture medium comprises myo-inositol.

Typically the glutathione is isolated from the culture medium.

According to a preferred aspect of this embodiment, the process is a process according to the invention utilizing a mutant yeast strain as described above.

Typically, where myo-inositol is included in the culture medium in processes of the invention, the concentration of myo-inositol is from about 0.01 mM to 100 mM (1.8 mg/L to 18000 mg/L), more typically about 0.1 to 10 mM, more typically from about 0.2 to 5 mM, even more typically from about 0.5 to 2 mM, and more typically about 1 mM.

A synergistic effect of myo-inositol and elevated levels of carbon source, such as glucose or the resulting ethanol, on the production of glutathione by yeast cells has also been found.

Therefore, according to a preferred aspect of the processes of the invention, the culture medium comprises myo-inositol and elevated levels of a carbon source.

Typically the carbon source is selected from fermentable sugars, more typically glucose or fructose or a combination thereof, and/or from oligosaccharides which are homo- or hetero-oligomers comprising fermentable sugar moieties, such as sucrose or maltose, even more typically sucrose. Other mono- and oligosaccharides (such as galactose, xylose, lactose, glucosyl sucrose oligosaccaharides such as raffinose and stachyose) or sugar alcohols (such as mannitol, xylitol) are also contemplated where yeast strains are capable of utilizing these sugars.

Alternatively, the carbon source may be a non-fermentable carbon source, more typically ethanol. The ethanol may be added as such to the other culture medium ingredients or, more typically, result from the fermentation of sugars by the yeast culture.

Typically, a carbon source is included in a culture medium at a concentration of about 1-2% w/v. Where elevated carbon source levels are to be included in the culture medium in combination with myo-inositol, the concentration of this substrate in the initial, uninoculated, culture medium, is typically greater than about 2% w/v, more typically between about 2% and 10% w/v, more typically between about 3% and 8% w/v; more typically between about 3% and 6% w/v, and even more typically about 4% w/v

According to a preferred aspect, the yeast strain is grown as a batch-wise culture. If desired, the glutathione may then be extracted from the culture medium by any of a number of known methods, such as chromatographic methods.

Alternatively, the yeast may be grown under continuous culture conditions, allowing for continuous harvesting of culture medium and therefore recovery of secreted glutathione.

According to yet another aspect of the process of the invention, the process relates to dough preparation. Methods of preparing doughs/baked products are well known in the art. Yeast is typically combined with the other dough components (typically flour, salt, shortening, bread improvers and other additives) as approximately 1-2% of flour weight, although this may vary depending on the type of dough and fermentation type (such as sponge-and-dough, rapid dough/mechanical dough preparation, high-sugar doughs). Although the mutant yeast may make up the total yeast component of the dough, it may also be added as a proportion only of the total yeast component of the dough, a standard commercial baker's yeast making up the remaining amount. Doughs prepared by such processes, or baked products derived therefrom are also provided.

According to yet another aspect of the process of the invention, the process is part of fermentation of a beverage, typically beer or wine. Antioxidants are routinely added to fermented beverages so as to inhibit oxidation of the alcohol (or other components)—a process according to this aspect provides the benefit of avoiding the need to add exogenous antioxidants to the brew. Processes for the production of fermented products are well known to those skilled in the art, and amounts of yeast to be added vary significantly amongst targeted products. The mutant strain may comprise all or a portion only of the total yeast component to be added.

Processes according to the invention for the production of glutathione will comprise any suitable technique known to those in the art. Typically the process will be carried out in fermenters, more typically industrial scale fermenters such as are already in use for the commercial production of baker's yeast.

For example, for batch-wise commercial production of glutathione, a seed culture of the mutant yeast will be produced for inoculation into a fermenter containing a suitable culture medium typically comprising from about 1-2% total fermentable sugars as well as a suitable nitrogen source (such as urea) and a phosphate source (such as monoammoniumphosphate) and optionally growth factors such as vitamins (for example, biotin), and/or such as a metabolite or growth factor which the mutant yeast strain is unable to synthesize or for which the mutant yeast strain has a restricted ability for synthesis. In the latter case, the metabolite/growth factor is maintained at sub-optimal concentrations in the fermentation medium, typically at about half-optimal levels. Typically, once ethanol production has ceased and the ethanol content of the culture medium has dropped to about 0.1-0.3% v/v, an exponential feeding protocol is started by increasing rate of feeding of a sugar source containing, typically, approximately 18-30% total fermentable sugars. The sugar feeding rate is kept at a rate whereby ethanol consumption predominantly exceeds ethanol production (except for the option of a sugar pulse, depending on the desired growth protocol and target activity of the yeast). A suitable nitrogen source and phosphate source are added in pre-determined amounts throughout the fermentation, the amounts depending on the final total yeast solids and the target protein content (typically between 40 to 60% Kjehldal protein). Metabolites and/or growth factors, if the mutant yeast strain is unable to synthesize one or more of these or has a restricted ability for the synthesis, will also be added throughout the fermentation at sub-optimal levels so as to maintain growth. Other additives, such as anti-foam are added if required.

Since intracellular glutathione has been found in these studies to overaccumulate prior to secretion, and in many of the strains tested, altered glutathione metabolism was triggered by amino acids limitation (particularly leucine, isoleucine and valine), growth of selected strains under a continuous state of low-leucine (or other parameters) is expected to provide a means of increasing glutathione production further. This could be achieved via the use of a continuous fed-batch culture system. This approach would maintain cells under optimal conditions to facilitate/maximise glutathione production.

2. Yeast Strains for Glutathione Production

The invention also relates to novel strains obtained by any form of directed mutagenesis, consisting of generating, preferably in industrial strains of yeasts, particularly baker's yeast, or in the starting haploids that served for construction of the industrial strains, mutations, monogenic or not, giving the required phenotype in the strains. This includes strains selected after conventional mutation treatment, for example using chemical/physical agents or molecular biological techniques or standard selection recombination methods to generate multiple mutants.

2.1 Yeast Mutants with Increased Glutathione Secretion

The present invention therefore also relates to a mutant yeast strain having at least two mutations selected from the following groupings, which may overlap:
i) mutation in a gene or genes encoding components of the mitochondrial respiratory chain or nuclear genes encoding proteins that maintain the integrity of the mitochondrial genome, or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26S proteosome;
viii) mutation in a gene or genes affecting transportation of glutathione across the yeast cell membrane;
ix) mutation in a gene or genes affecting glutathione degradation; and
x) mutation in a gene or genes involved in vacuolar function.

The yeast strain may have more than one mutation within one of the above groups (i) to (x). For example, two different mutations in group (ii) genes may be contemplated such as a combination of a mutation affecting glycerol synthesis and a mutation in a gene the expression of which suppress or result in competition for the GLN1, GLT1 glutamate synthesis pathway.

According to one aspect, the yeast is a mutant strain of Saccharomyces cerevisiae in which the genes encoding components of the mitochondrial respiratory chain or nuclear genes encoding proteins that maintain the integrity of the mitochondrial genome are selected from the following: YBL009W(ATP1); YBR003W(COX1); YBR037C (SCO1); YBR191W(RPL21α); YBR220C; YBR268W(MRPL37); YCR046C (IMG1); YDL069C (CBS1); YDL107W(MSS2); YDL202W(MRPL11); YDR079W(PET100); YDR175C(RSM24); YDR197W(CBS2); YDR204W(COQ4); YDR298C(ATP5); YDR322 W (MRPL35); YDR337W (MRPS28); YDR462 W (MRPL28); YDR529C (QCR7); YER017C(AFG3); YER141W(COX15); YER153C(PET122); YER154W (OXA1); YFL034W(MRPL7); YGR062C(COX18); YGR171C(MSM1); YGR220C (MRPL9); YGR257C; YHL004W(MRP4); YHL038C (CBP2); YHR011 W(DIA4); YHR051W(COX6); YHR120w(MSH1); YHR147C(MRPL6); YIL006W; YIL018W (RPL2B); YIL065C(FIS1); YIL070C (MAM33); YIL093C(RSM25); YIL098C (FMC1); YIR021W(MRS1); YJL063C(MRPL8); YJL102W(MEF2); YJL166W (QCR8); YJL209W(CBP1); YJR144W(MGM101); YKL003C (MRP17); YKL032C (IXR1); YKR006C(MRPL13); YLL009C(COX17); YLL018C-A (COX19); YLR067C (PET309); YLR139C(SLS1); YLR295C(HSP60); YLR369W(SSQ1); YML078W (CPR3); YMR064W(AEP1); YMR072W(ABF2); YMR150C (IMP1); YMR193W (MRPL24); YMR228W(MTF1); YNL177C; YNR036C; YNR037C(RSM19); YNR045W (PET494); YOL009C (MDM12); YOL033W(MSE1); YOL095C (HMI1); YOR026W (BUB3); YPL132W(COX11); YPL183W-A; YPR004C; YPR166C(MRP2); and YPR191 W(QCR2). Mutations which result in mitochondrial respiratory deficiency (petite mutations) are also contemplated.

According to another aspect, the yeast is a mutant strain of Saccharomyces cerevisiae in which the genes affecting the levels of NADH and NAD⁺ are selected from genes encoding enzymes which catalyze the synthesis of glycerol, ethanol and/or genes the expression of which suppress or result in competition for the GLN1, GLT1 glutamate synthesis pathway. Typically these genes are selected from YIL053W(RHR2), YOR375C (GDH1) and YNL229c (URE2).

According to another aspect, the yeast is a mutant strain of Saccharomyces cerevisiae in which the genes affecting the assimilation and metabolism of nitrogen in the cell are selected from: YDR300C(PRO1); YDR448W(ADA2); YEL009C(GCN4); YEL062W(NPR2); YGL227W(VID30); YGR252W(GCN5); YNL106C (INP52); YNL229C (URE2); YOR375C (GDH1); and YPL254W(HFI1).

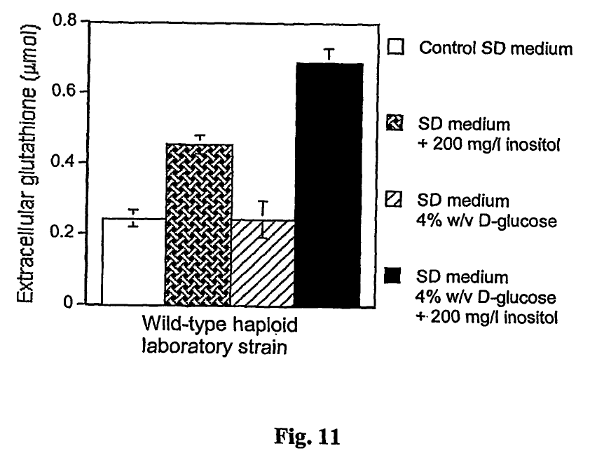

According to another aspect, the yeast is a mutant strain of Saccharomyces cerevisiae in which the genes encoding regulatory components of the Ras/cAMP/PKA pathway or otherwise affecting the activity of the Ras/cAMP/PKA pathway are selected from YOL081W(IRA2); YOR360C(PDE2); and YNL098C(RAS2; RAS2Val19dominant mutation—see FIG. 11).

According to another aspect, the yeast is a mutant strain of Saccharomyces cerevisiae in which the genes affecting endosomal function are selected from: YCL008C (VPS23; STP22); YDR456W(NHX1); YJR102C(VPS25); YKL002W(DID4); YKL041W(VPS24); YKR035W-A (DID2); YLR025W(VPS321SNF7); YLR119W (SRN21VPS37); YLR417W(VPS36); YMR077C(VPS20); YNR006W(VPS27); YPL065W(VPS28); and YPR173C (VPS4). Particularly those genes defined as class E compartment genes (Class E vps genes) are of interest.

According to another aspect, the yeast is a mutant strain of Saccharomyces cerevisiae in which the genes affecting endoplasmic reticulum function, the Golgi to endosome to vacuole transportation pathway or vacuolar function are selected from: YFL031 W(HAC1), YDR027C (LUV11VPS54); YDR323C (PEP7/VPS19); YDR484W (VPS52/SAC2); YBR131W(CCZ1); YDR486C(VPS60); YHR012W(VPS29); YJL154C (VPS35); YLR1148W(VAC1/PEP31VPS18); YML001W(YPT7); and YOR036W (PEP12/VPS6).

According to another aspect, the yeast is a mutant strain of Saccharomyces cerevisiae in which the genes affecting ubiquitin levels and ubiquitin-mediated proteolysis via the 26S proteosome are selected from: YBR173C (UMP1); YER151C (UBP3); YFR010W(UBP6); YHL011C(PRS3); YKL213C(DOA1); YNR051C(BRE5); YPL003W(ULA1); and YPL074W(YTA6).

According to another aspect, the yeast is a mutant strain of Saccharomyces cerevisiae in which a gene involved in transportation of glutathione across the yeast cell membrane is YDR135C (YCF1) or YJL212C (HGT1).

According to another aspect, the yeast is a mutant strain of Saccharomyces cerevisiae in which a gene involved in glutathione degradation is pep3, pep12, or pep7.

According to another aspect, the yeast is a mutant strain of Saccharomyces cerevisiae in which a gene involved in vacuolar function is pep3, pep12, or pep7.

According to a preferred aspect, the yeast strain has mutations in two or more of groups (i) to (x) listed above and will also be a mutant for the synthesis of one or more metabolites and/or essential growth factors, wherein the mutant is unable to synthesize said one or more metabolites and/or essential growth factors or has a restricted ability for synthesis of said one or more metabolites and/or essential growth factors. Typically the one or more metabolites and/or essential growth factors are amino acids or precursors or metabolites thereof. More typically the one or more metabolites and/or essential growth factors are selected from leucine, isoleucine or valines or precursors or metabolites thereof, and even more typically from leucine or precursors or metabolites thereof.

Double mutants which are contemplated by the present invention include, but are not restricted to:
endosomal function (Class E vps or other protein sorting) mutation (as defined in group (v) above)+Ras/c-AMP/PKA mutation (as defined in group (iv) above), examples being: ykl002w (did4)+yor360c (pde2); ykl002w (did4)+yol081w (ira2); and ykl002w (did4)+RAS2val19;
mutation affecting vacuolar function or Golgi to endosome to vacuole transport (as defined in group (vi) above)+Ras/c-AMP/PKA pathway mutation (as defined in group (iv) above), examples being: ylr1148w (vac1/pep31vps18)+yor360c (pde2); ylr1148w (vac1/pep31vps18)+yol081w (ira2); ylr1148w; (vac1/pep31vps18)+RAS2val19; yor036w (pep12/vps6)+yor360c (pde2); yor036w (pep12/vps6)+yol081w (ira2); and yor036w (pep12/vps6)+RAS2val19;
endosomal function (Class E vps or other protein sorting) mutation (as defined in group (v) above)+nitrogen assimilation pathway mutation (as defined in group (iii) above), examples of the latter mutation class being: ydr300c (pro1); ydr448w (ada2); yel009c (gcn4); yel062w (npr2); ygl227w (vid30); ygr252w (gcn5); ynl106c (inp52); ynl229c (ure2); yor375c (gdh1); and ypl254w (hfi1), and examples of some such crosses being: ykl002w (did4)+ynl229c (ure2); ykl002w (did4)+yor375c (gdh1); and ykl002w (did4)+ydr300c (pro1);
endosomal function (Class E vps or other protein sorting) mutation (as defined in group (v) above)+mutation that affects NADH levels (as defined in group (iii) above), an example being ykl002w (did4)+yil053w (rhr2);
endosomal function (Class E vps or other protein sorting) mutation (as defined in group (v) above)+mitochondrial mutation (as defined in group (i) above), examples being: ykl002w (did4)+ykl003c (mrp17) and other mitochondrial respiratory chain mutants such as ykl002w (did4)+ypr004c; ykl002w (did4)+yhr011w (dia4);
endosomal function (Class E vps or other protein sorting) mutation (as defined in group (v) above)+mutation in ubiquitin mediated protein degradation (as defined in group (vii) above), an example being ykl002w (did4)+ykl213c (doa1); and
endosomal function (Class E vps or other protein sorting) mutation (as defined in group (v) above)+glutathione transport mutant (as defined in group (viii) above), an example being ykl002w (did4)+yjl212c (opt1/hgt1);
Ras/c-AMP/PKA pathway mutation (as defined in group (iv) above)+mitochondrial mutation (as defined in group (i) above), an example being yor360c (pde2)+ykl003c (mrp17);
Ras/c-AMP/PKA pathway mutation (as defined in group (iv) above)+glycerol biosynthesis/NADH metabolism mutation (at the same time increasing GLT1 and GLN1 activity, as defined in group (iii) above), an example being yor360c (pde2)+yil053w (rhr2);
Ras/c-AMP/PKA pathway mutation (as defined in group (iv) above)+nitrogen assimilation pathway mutation (as defined in group (iii) above), examples being: yor360c (pde2)+ynl229c (ure2); yor360c (pde2)+yor375c (gdh1); and yor360c (pde2)+ydr300c (pro1);
Ras/c-AMP/PKA pathway mutation (as defined in group (iv) above)+ubiqitin mutation (as defined in group (vii) above), an example being yor360c (pde2)+ykl213c (doa1); and
mitochondrial mutation (as defined in group (i) above)+nitrogen assimilation pathway mutation (as defined in group (iii) above, an example being ykl003c (mrp17)+ynl229c (ure2);
mitochondrial/petite mutation as defined in group (i) above+glutathione transport mutant (as defined in group (viii) above), an example being ?+yjl212c (opt1/hgt1).
mutation affecting endoplasmic reticulum function as defined in group (iv) above+glutathione transport mutation as defined in group (viii) above, an example being BSO4 mutation (yfl031w (hac1))+ydr135c (ycf1). Mutants with three or more mutations are also contemplated by the present invention and may include, but are not restricted to: did4+pde2+ure2; did4+pde2+ure2+mrp17; and pde2+glycerol mutant+ure2.

Other multiple mutants which are contemplated by the present invention are yeast strains having at least one mutation selected from groups (i) to (x) as described above, in addition to genetic manipulation resulting in overexpression of the glutathione synthesis pathway. Such manipulations resulting in, for example, overexpression of gammaglutamylcysteine synthetase (GSH1), glutathione synthetase (GSH2) or GSH1 and GSH2.

Some of the mutants described above can also be grouped by reference to their glutathione secretion in response to external pH, or in response to amino acid availability (particularly availability of the branched chain amino acids leucine, isoleucine and valine), or their ability to utilise glutathione as a sole nitrogen source (cells defective in glutathione degradation and/or transport). Several of the mutants described herein have been found to oversecrete glutathione due to defects in glutathione degradation. This was tested by their ability to grow using glutathione as a sole nitrogen source. Although a failure to grow under these conditions could also result from blocked uptake of glutathione, this is less likely since these cells overaccumulate GSH intracellularly prior to secretion (when they are grown on standard SD medium). They are also hypersensitive to thiol-specific reducing agent dithiothreitol (DTT) indicating that their primary defect is a failure to degrade excess cytoplasmic glutathione.

Strains having two or more mutations selected within each of, or amongst the groupings described above are also contemplated according to the invention. Particularly, it is envisaged that, for example, a double mutant generated with the combination of: a leucine more-responsive mutation and a leucine less-responsive mutation; a pH more-responsive and a pH less-responsive mutation; or of two different glutathione utilization/transportation defective mutations; may produce a strain with greater glutathione production and/or secretion than either of the single mutants.

Although, for the most part, glutathione secretion by other mutants investigated was highly dependent on external pH, examples of mutants with glutathione secretion less dependent on external pH are: yjl153c (inol); yol108c (ino4); and ylr226w (bur2).

Examples of mutants with glutathione secretion highly dependent on branched chain amino acid availability are: ynl229c (ure2); yhl023c; yol138c; yel062w (npr2); yol027c; ylr119w (vps37); yol050c; yjl056c (zap1); ybr003w (cox1); ynr005c; ycl008c (vps23); yjr102c (vps25); yor375c (gdh1); yol004w (sin3); ydr486c (vps60); ydr276c (pmp3); yjl188c (bud19); ylr417w (vps36); ykl002w (vps2); ykr035w-a (did2); ypr004c; ylr025w (vps32); yfr010w (ubp6); and ykl213c (doa1).

Examples of mutants with glutathione secretion less dependent on branched chain amino acid availability are: ylr1148w (pep3); ylr396c (vps33); yor036w (pep12); ydr323c (pep7); ydr027c (luv1); ydr484w (sac2); yfr019w (fab1); ykr001c (vps13); ydr495c (vps3); ynl297c (mon2); yor070c (gyp1); yjl102w (mef2); yol081w (ira2); yjl153c (ino1); yol108c (ino4); ylr114c (efr4); yjl095w (bck1); yhr030c (mpk1); ydr264c (akr1); yjl042w (mhp1); yal047c (spc72); ycl007c (cwh36); ydl074c (bre1); yer116c (slx8); ynr036c; ybr056w; yjl176c (swi3); and yil029c.

Examples of mutants which are likely to be defective in glutathione degradation and/or transport are:

| | Common | |
|---|---|---|
| Locus | name | Function |
| yol081w | ira2 | GTPase-acting protein for Ras1p & Ras2p |
| ynl229c | ure2 | Regulator nitrogen catabolite repression |
| yjr102c | vps25 | ESCRT-II complex |
| ylr417w | vps36 | ESCRT-II complex |
| ypl002c | vps22 | ESCRT-II complex |
| ykl002w | vps2 | ESCRT-III complex |
| ylr025w | vps32 | ESCRT-III complex |
| ymr077c | vps20 | ESCRT-III complex |
| ykr035w-a | did2 | Endosomal protein sorting |
| ypr173c | vps4 | AAA-ATPase of ESCRT complexes |
| ydr027c | luv1 | Subunit (Sac2p-Vps53p-Luv1p) complex |
| ydr484w | sac2 | Subunit (Sac2p-Vps53p-Luv1p) complex |
| ydr323c | pep7 | FYVE domain-containing Vac. Inherit |
| ydr495c | vps3 | Vacuolar sorting protein and segregation |
| ygl227w | vid30 | Vacuolar import and degradation |
| yil017c | vid28 | Vacuolar import and degradation |
| yll040c | vps13 | Protein involved in vacuolar sorting |
| ylr1148w | pep3 | Class C complex, vacuolar biogenesis |
| ylr396c | vps33 | Class C complex, vacuolar biogenesis |
| yml097c | vps9 | Protein involved in vacuolar sorting |
| yor036w | pep12 | SNARE-Syntaxin of the late endosome |
| ygl124c | mon1 | Vacuolar protein sorting |
| ygl223c | cod3 | Component of Sec34p-Sec35p complex |
| ykl212w | sac1 | Phosphoinisotide phosphatase |
| yer151c | ubp3 | Ubiquitin-specific protease |
| yal047c | spc72 | Cytoplasmic plaque of spindle pole body |
| ycl007c | cwh36 | Generation of mannoprotein layer |
| yhr030c | mpk1 | Serine/threonine protein kinase |
| yjl095w | bck1 | Serine/threonine protein kinase |
| ynl225c | | Component of spindle pole body |
| yor043w | whi2 | DNA repair protein |
| ybr036c | csg2 | Ca²⁺ homeostasis protein (CHP) family |
| ygr217w | cch1 | Voltage-gated Ca²⁺ channel |
| ybr279w | paf1 | Protein associated with RNA polymerase II |
| ybr289w | snf5 | Component of SWI-SNF complex |
| ydr448w | ada2 | Component of SAGA & ADA complexes |
| ygr252w | gcn5 | Component of SAGA & ADA complexes |
| ylr226w | bur2 | Regulation of transcription |
| yol004w | sin3 | Component of histone deacetylase B |
| ypl254w | hfi1 | Component of the ADA complex |
| ydr264c | akr1 | Pheromone signaling pathway |
| yil053w | rhr2 | D,L-glycerol phosphate phosphatase |
| ynl280c | erg24 | C-14 sterol reductase |
| ypl022w | rad1 | Nucleotide excision repairosome |
| yal024c | lte1 | Required for termination of M phase |
| ydl023c | | Protein of unknown function |
| ygl107c | | Protein of unknown function |
| yil029c | | Protein of unknown function |
| yil041w | | Protein of unknown function |
| yil077c | | Protein of unknown function |
| yil097w | fyv10 | Protein of unknown function |
| yil11Ow | | Protein of unknown function |
| ymr123w | pkr1 | Protein of unknown function |
| yol027c | | Protein of unknown function |

Many of the mutants listed above are defective in vacuolar function, where glutathione degradation is known to occur. Glutathione breakdown is therefore a mechanism that leads to increased glutathione production.

Double mutants such as ure2pep2, ure2 inol, inol pep3, vps22 inol, ure2 vps37, inol vps37, amongst others, are contemplated by the present invention.

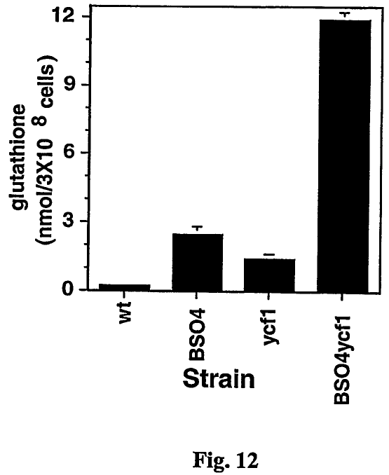

The present invention also relates to a yeast double mutant strain herein described as BSO4ycf1 (the glutathione secretion of which, relative to the single ras2 mutation or the parental strain, is illustrated in FIG. 12: growth conditions, media and timing as described example 1). The BSO4 mutation has been detected as a defect in the HAC1 gene.

The present invention also relates to a method of preparing a dough comprising dough components with a yeast strain according to the invention. Doughs prepared by this method, and baked products derived therefrom, are also provided.

The present invention also relates to a method of producing a fermented product comprising adding to the unfermented precursor component(s) of said product a yeast strain according to the invention. Fermented products obtained by this method are also provided.

2.2 Generation of Mutants

Generation of mutant strains according to the invention and/or for use in processes according to the invention may be generated by any one of a wide range of methods known to those of skill in the art and such as are described in well known texts such as “Methods in Yeast Genetics” (1997) (Alison Adams, Daniel E. Gottschling, Chris A. Kaiser, Tim Stearns, eds., Cold spring Harbour Laboratory Press) and “Molecular Cloning”, 2nd Edition (1989) (Sambrook, J., E. F. Fritsch and T. Maniatis, eds., Cold Spring Harbor Laboratory Press, Cold Spring Harbor, N.Y. 1989).

Typically, the mutational techniques may include any method which results in a mutation which results in a “functional” deficiency, irrespective of how the genes have been mutated. Mutations may typically include deletion mutations, point mutations, insertion or substitution mutations, frame-shift mutations or any other method that results in inactivation of a gene (including RNAi approaches to selectively inactivating gene expression) or chemical/physical means. Suitable techniques may include mutagenic techniques (using mutagens such as UV, X-ray, γ-ray, ethylmethanesulfonate, N-methyl-N′-nitro-N-nitrosoguanidine) or recombinant DNA techniques, chemical/physical agents, molecular biological techniques (including PCR methods to generate deletants, site directed mutagenesis protocols), or standard selection recombination methods to generate multiple mutants. Multiple mutations can be generated either by successive application of mutagenic techniques or by recombination of single mutations of strains using standard hydridization techniques involving mating, diploid isolation, sporulation and recombination, or by processes of recombination.

2.3 Compositions Comprising Glutathione Produced by the Process of the Invention, and Uses Thereof.

The present invention also relates to a glutathione obtained by the process of the invention. The glutathione may be provided as a concentrated form of the culture medium or it may be purified to a desired degree.

The glutathione may be used in a wide variety of applications as a catalyst, reactant or reductant/antioxidant. Fields of application include, but are not restricted to personal health care, pharmaceuticals, nutraceuticals, cosmetics, food (including bakery and fermentation technology) and animal feeds, agriculture, aquaculture, paints, and fermentation media. For pharmaceutical purposes the glutathione is preferably provided as a purified compound, typically greater than 60% pure, more typically greater than 70% pure, more typically greater than 80% pure, even more typically greater than 90% pure, and more preferably greater than 95% pure.

Thus, the present invention also relates to a personal health care composition comprising glutathione obtained by the process of the invention and a pharmaceutically or topically acceptable carrier.

The present invention also relates to a pharmaceutical composition comprising glutathione obtained by the process of the invention and a pharmaceutically acceptable carrier. Such pharmaceutical compositions may be used in the treatment of, for example, cancer, cardiovascular disease (such as atherosclerosis), oxidative damage to tissue (such as aging, or progressive protein oxidation in the eye lens), respiratory distress syndrome, toxicology, AIDS, and liver disease.

The present invention also relates to a food or nutraceutical composition comprising glutathione obtained by the process of the invention in combination with one or more food components. The food/nutraceutical composition may be selected from liquids, semi-solids and solids.

The present invention also relates to a dough or bread improving composition comprising glutathione obtained by the process of the invention and a suitable carrier. The carrier may be selected from a wide variety of bakery acceptable ingredients, including flour and/or sugar and the composition may also include other bread improving ingredients such as enzymes (including cellulases, glucanases, amylases, xylanases, arabinoxylanases, dextrinases, maltases, etc.). The composition may be in the form of a powder, granulate or liquid.

The present invention also relates to an animal feed additive comprising glutathione obtained by the process of the invention and a suitable carrier. The carrier may be selected from a wide variety of acceptable animal feed ingredients, such as flour (including wheat, corn or soy), and the composition may also include other animal feed additives including those which improve the digestibility of the food such as enzymes (including cellulases, glucanases, amylases, xylanases, arabinoxylanases, dextrinases, maltases, etc.). The composition may be in the form of a powder, granulate or liquid.

The present invention also relates to an animal health care composition comprising glutathione obtained by the process of the invention and a veterinary acceptable carrier.

The present invention also relates to a method for preventing oxidative damage in the circulation or tissues of a mammal, said method comprising administering to said mammal an effective amount of a composition comprising glutathione obtained by the process of the invention.

The present invention also relates to a method of protecting a food product from oxidative deterioration comprising adding to said food product an effective amount of glutathione obtained by the process of the invention or a composition comprising it. Food products prepared by said method are also provided. The food product may be liquid, semi-solid or solid.

The present invention also relates to a method of preparing a dough comprising combining dough components with an effective amount of glutathione obtained by the process of the invention. Doughs prepared by this method, or baked products derived therefrom are also provided.

### EXAMPLES

### Example 1

### Identification of Yeast Mutants for Glutathione Production

A deletion library of yeast strains derived from yeast strain BY4743, and as described in Winzeler E. A. et al., (1999), Science 285: 901-906 was purchased from EUROSCARF Saccharomyces cerevisiae (www.rz.unifrankfurt.de/FB/fbl6/mikro/euroscarf). According to the Winzeler E. A. et al reference, these mutants are deletion strains according to the following procedure: two long oligonucleotide primers are synthesized, each containing (3 [prime] to 5 [prime]) 18 or 19 bases of homology to the antibiotic resistance cassette, KanMX4 (U1, D1), a unique 20-bp tag sequence, an 18-bp tag priming site (U2 or D2), and 18 bases of sequence complementary to the region upstream or downstream of the yeast ORF being targeted (including the start codon or stop codon; see http://sequence-www.stanford.edu/group/yeast/yeast_deletion_project/new_deletion strategy.html). These 74-mers are used to amplify the heterologous KanMX4 module, which contains a constitutive, efficient promoter from a related yeast strain. Ashbya gosspii, fused to the kanamycin resistance gene, npt1. Because oligonucleotide synthesis is 3[prime] to 5[prime] and the fraction of full-size molecules decreases with increasing length, improved targeting is achieved by performing a second round of PCR using primers bearing 45 bases of homology to the region upstream and downstream of a particular ORF. Transformation with the PCR product results in replacement of the targeted gene upon selection for G418 resistance. The unique 20-mer tag sequences are covalently linked to the sequence that targets them to the yeast genome, creating a permanent association and genetic linkage between a particular deletion strain and the tag sequence.

The mutants were screened for glutathione production, both intracellular and extracellular after growth in the following medium and under the following conditions.

Growth Medium (SD minimal medium)
(mass per litre water)

| | | |
|---|---|---|
| D-glucose | 20 | g |
| ammonium sulphate | 5 | g |
| yeast nitrogen base | 1.7 | g |
| (without amino acids, without ammonium sulphate) | | |
| (Purchased from Difco) | | |

Additional Growth Supplements

| | |
|---|---|
| L-leucine | O.131 g |
| L-isoleucine | 0.066 g |
| L-valine | 0.059 g |
| L-histidine | 0.209 g |
| uracil | 0.022 g |

The medium was sterilised by autoclaving at 121° C. for 15 min. The additional supplements leucine, isoleucine, valine, histidine and uracil were prepared separately as a sterile 100× stock solution and were added to the growth medium after autoclaving to give the final quantity per litre of each ingredient shown above.

### Growth Parameters/Conditions

Culture vessel: Standard 24 well flat bottomed plastic culture plate manufactured by Sarstedt
Growth temperature 30° C.

Growth period 48h
Orbital shaker speed 500 rpm
Volume of growth medium per culture plate well 1 ml
Inoculating cultures were pre-grown in the above medium for 48h and were used to inoculate 24 well cultures containing the same medium at a starting culture density of approximately 2×10⁴ cells per ml.

### Growth Conditions

The sterilised medium was aliquoted into 24 well plastic culture plates and inoculated via the addition of the appropriate inoculating culture. The cultures were shaken (500 rpm) at 30° C. for 48h and the optical density of the culture was measured at 600 nm. A 500 microlitre aliquot of each culture was transferred to a 1.5 ml Eppendorf microcentrifuge tube which was centrifuged for 30 seconds at 1000 g. A 100 microlitre of the clarified culture medium was taken to allow quantification of extracellular glutathione content. Intracellular and extracellular glutathione were determined by a method adapted from that reported by Vandputte C. et al., Cell Biology and Toxicology (1994) vol 10: 415-421:

Sample Preparation

1. Spin down cells for 30s at 1000 g (4° C.) for culture up to 1 mL or for larger cultures (10-50 ml) spin down cells 5mim at 500 rpm in an SS34 rotor (4° C.).

2. Take a sample of the culture medium for extracellular glutathione quantification using the protocol described later in this section.

3. For the quantification of intracellular GSH was the pellet with an equal volume (equal to the harvest volume) of ice-cold PBS pH 7.4 and centrifuge as above.

4. Lyse the cell pellet by the addition of 400 ul ice-cold 1.3% (w/v) 5-sulfosalicylic acid/8 mM hydrochloric acid (4° C.) and add glass beads to facilitate breakage using a mini bead beater (breakage time 1 min ant high speed) or vortex vigorously for 2 min.

5. Centrifuge the cell lysate for 5 min at 8000 g (4° C.) to clarify solution. The sample is ready to assay.

6. Dilute sample as required

Assay Reaction Mixture Contents

143 mM NaH₂PO₄
6.3 mM EDTA pH 7.4
400 mg/L 5,5′-dithion-bis(2-nitrobenzoic acid)
100 mg/L NADPH
   Glutathione Assay Procedure

Add as 4 parts reaction mixture per 1 part unknown/sample.

Start the reaction by the addition of 40 microlitres of 0.85units/ml glutathione reductase enzyme (purchased from Sigma chemical Company).

Monitor the reaction at 410 nm wavelength. Compare the change in absorbance to suitably prepared standards containing a known quantity of glutathione. Compare the quantity of glutathione produced divided by the number of cells isolated in the sample. Following normalisation of the data in this way GSH levels may be compared between strains. Typically glutathione values should be compared for both raw concentrations as well as for concentration normalised to cell number.

Glutathione levels produced by respective cultures were adjusted to culture density and then compared to the figure recorded for the wild type/parent strain grown under identical conditions.

Yeast strains having the following gene deletions were found to provide elevated accumulation of extracellular glutathione, and the results are also provided in Tables 1 to 10.

| | | | | | |
|---|---|---|---|---|---|
| yal002w | ydr300c | ygr062c | yjl042w | ylr262c | yol004w |
| yal024c | ydr322w | ygr100w | yjl053w | ylr268w | yol008w |
| yal047c | ydr323c | ygr105w | yjl063c | ylr295c | yol009c |
| yar002c-a | ydr332w | ygr150c | yjl095w | ylr322w | yol018c |
| ybl007c | ydr337w | ygr171c | yjl102w | ylr330w | yol027c |
| ybl009w | ydr448w | ygr217w | yjl138c | ylr342w | yol033w |
| ybl027w | ydr456w | ygr220c | yjl152w | ylr357w | yol050c |
| ybl100c | ydr462w | ygr252w | yjl154c | ylr360w | yol081w |
| ybr003w | ydr475c | ygr257c | yjl166w | ylr369w | yol095c |
| ybr036c | ydr484w | ygr284c | yjl176c | ylr373c | yol108c |
| ybr037c | ydr486c | yhl004w | yjl183w | ylr396c | yol138c |
| ybr041w | ydr495c | yhl011c | yjl188c | ylr417w | yor008c |
| ybr056w | ydr497c | yhl023c | yjl201w | ylr439w | yor026w |
| ybr059c | ydr518w | yhl025w | yjl204c | ylr447c | yor036w |
| ybr125c | ydr529c | yhl031c | yjl209w | yml001w | yor043w |
| ybr127c | ydr533c | yhl038c | yjl212c | yml048w | yor069w |
| ybr131w | yel007w | yhr010w | yjr059w | yml071c | yor070c |
| ybr162c | yel009c | yhr011w | yjr063w | yml078w | yor088w |
| ybr163w | yel036c | yhr012w | yjr075w | yml097c | yor089c |
| ybr173c | yel051w | yhr030c | yjr102c | ymr004w | yor106w |
| ybr191w | yel062w | yhr051w | yjr144w | ymr064w | yor132w |
| ybr220c | yer017c | yhr116w | ykl002w | ymr066w | yor332w |
| ybr268w | yer056c | yhr120w | ykl003c | ymr072w | yor360c |
| ybr279w | yer116c | yhr129c | ykl032c | ymr077c | yor375c |
| ybr289w | yer122c | yhr147c | ykl041w | ymr123w | yor384w |
| ycl007c | yer141w | yhr171w | ykl212w | ymr150c | ypl003w |
| ycl008c | yer151c | yhr185c | ykl213c | ymr151w | ypl017c |
| ycr046c | yer153c | yil006w | ykr001c | ymr193w | ypl022w |
| ydl023c | yer154w | yil008w | ykr006c | ymr228w | ypl037c |
| ydl039c | yfl031w | yil017c | ykr035c | ynl098c | ypl058c |
| ydl069c | yfl034w | yil018w | ykr035w-a | ynl106c | ypl065w |
| ydl074c | yfr010w | yil029c | ykr054c | ynl177c | ypl074w |
| ydl077c | yfr019w | yil041w | yll009c | ynl215w | ypl091w |
| ydl107w | ygl025c | yil053w | yll010c | ynl225c | ypl120w |
| ydl191w | ygl107c | yil065c | yll018c-a | ynl229c | ypl132w |
| ydl202w | ygl115w | yil070c | yll040c | ynl280c | ypl149w |
| ydr017c | ygl124c | yil077c | ylr006c | ynl296w | ypl183w-a |
| ydr027c | ygl127c | yil092w | ylr025w | ynl297c | ypl254w |
| ydr079w | ygl167c | yil093c | ylr067c | ynr005c | ypr004c |
| ydr175c | ygl168w | yil097w | ylr114c | ynr006w | ypr036w |
| ydr197w | ygl212w | yil098c | ylr119w | ynr036c | ypr099c |
| ydr200c | ygl227w | yil110w | ylr139c | ynr037c | ypr100w |
| ydr204w | ygl237c | yir021w | ylr148w | ynr045w | ypr159w |
| ydr264c | ygl244w | yjl004c | ylr193c | ynr050c | ypr166c |
| ydr276c | ygl252c | yjl022w | ylr226w | ynr051c | ypr173c |
| ydr298c | ygr021w | yjl029c | ylr261c | yol001w | ypr191w |

TABLE 1
Mitochondrial related mutations - ratio of glutathione secretion
by mutant to amount secreted by parental strain

| | | Ratio GSH |
|---|---|---|
| | | secreted |
| Mitochondrial related | | mutant:parental |
| ybl009w | atp1 | 6 |
| ybr003w | cox1 | 12 |
| ybr037c | SC01 | 10 |
| ybr191w | rpl21a | 4 |
| ybr220c | | 7 |
| ycr046c | img1 | 8 |
| ydl069c | cbs1 | 10 |
| ydl107w | mss2 | 8 |
| ydr079w | pet100 | 11 |
| ydr175c | rsm24 | 8.6 |
| ydr197w | cbs2 | 10 |
| ydr204w | coq4 | 8.6 |
| ydr298c | atp5 | 10.2 |
| ydr322w | mrpl35 | 9.7 |
| ydr462w | mrpl28 | 11 |
| ydr529c | qcr7 | 10 |
| yer141w | cox15 | 11 |
| yer153c | pet122 | 9 |
| yer154w | oxa1 | 11 |
| ygr062c | cox18 | 5.5 |
| ygr171c | msm1 | 11 |
| ygr220c | mrpl9 | 1.2 |
| ygr257c | | 14 |
| yhl004w | mrp4 | 10 |
| yhr011w | dia4 | 15.2 |
| yhr051w | cox6 | 7.1 |
| yhr120w | msh1 | 10.6 |
| yhr147c | mrpl6 | 11 |
| yil006w | | 6 |
| yil018w | rpl2b | 4 |
| yil065c | fis1 | 10.3 |
| yil070c | mam33 | 11 |
| yil093c | rsm25 | 6.8 |
| yil098c | fmc1 | 14 |
| yir021w | mrs1 | 8.6 |
| yjl063c | mrpl8 | 6.4 |
| yjl102w | mef2 | 7.8 |
| yjl166w | qcr8 | 7.8 |
| yjl209w | cbp1 | 8.3 |
| yjr144w | mgm101 | 11 |
| ykl003c | mrp17 | 29 |
| ykl032c | ixr1 | |
| ykr006c | mrpl13 | 8.1 |
| yll009c | cox17 | 2 |
| yll018c-a | cox19 | 10 |
| ylr067c | pet309 | |
| ylr139c | sls1 | |
| ylr295c | hsp60 | 1 |
| ylr369w | ssq1 | |
| yml078w | cpr3 | |
| ymr064w | aep1 | 10.8 |
| ymr072w | abf2 | 9.6 |
| ymr150c | imp1 | 8.1 |
| ymr193w | mrpl24 | 11.8 |
| ymr228w | mtf1 | 8.1 |
| ynl177c | | 7.6 |
| ynr036c | | 11 |
| ynr037c | rsm19 | 3.8 |
| ynr045w | pet494 | 10 |
| yol009c | mdm12 | |
| yol033w | mse1 | 8.7 |
| yol095c | hmi1 | 9.5 |
| yor026w | bub3 | 5 |
| ypl132w | COX11 | 8.4 |
| ypl183w-a | | 12 |
| ypr004c | | 24.5 |
| ypr166c | mrp2 | 8.4 |
| ypr191w | qcr2 | 7.1 |

TABLE 2
Ubiquitin related mutations - ratio of glutathione secretion
by mutant to amount secreted by parental strain

| | |
|---|---|
| | Ratio GSH secreted |
| Ubiquitin related | mutant:parental |

| | | |
|---|---|---|
| ybr173c | ump1 | 9.7 |
| yer151c | ubp3 | 3 |
| yfr010w | ubp6 | 18 |
| yhl011c | prs3 | 8.2 |
| ykl213c | doa1 | 25 |
| ynr051c | bre5 | 9 |
| ypl003w | ula1 | 35 |
| ypl074w | yta6 | 11 |

TABLE 3
Nitrogen assimilation related mutations - ratio of glutathione
secretion by mutant to amount secreted by parental strain

| | |
|---|---|
| | Ratio GSH secreted |
| Nitrogen related | mutant:parental |

| | | |
|---|---|---|
| ydr300c | pro1 | 18 |
| ydr448w | ada2 | 1.4 |
| yel009c | gcn4 | 1.7 |
| yel062w | npr2 | 6 |
| ygl227w | vid30 | 2.6 |
| ygr252w | gcn5 | 6.8 |
| ynl106c | inp52 | 1.3 |
| ynl229c | ure2 | 23.7 |
| yor375c | gdh1 | 5 |
| ypl254w | hfi1 | 5.1 |

TABLE 4
c-AMP related mutations - ratio of glutathione secretion
by mutant to amount secreted by parental strain

| | |
|---|---|
| | Ratio GSH secreted |
| c-AMP related | mutant:parental |

| | | |
|---|---|---|
| yol081w | ira2 | 25 |
| yor360c | pde2 | 22 |

TABLE 5
Cell wall related mutations - ratio of glutathione secretion
by mutant to amount secreted by parental strain

| | |
|---|---|
| | Ratio GSH secreted |
| cell wall | mutant:parental |

| | | |
|---|---|---|
| ydr017c | kcs1 | 7.3 |
| ydr497c | itr1 | 1.7 |
| yhr030c | mpk1 | 5 |
| yjl095w | bck1 | 2.4 |
| yjl152w | ino1 | 14 |
| ylr330w | chs5 | 5 |
| yol108c | ino4 | 13.1 |
| ypr159w | kre6 | 4.8 |

TABLE 6
Signal transduction related mutations - ratio of glutathione
secretion by mutant to amount secreted by parental strain

| | |
|---|---|
| | Ratio GSH secreted |
| Signal Transduction | mutant:parental |

| | | |
|---|---|---|
| yal024c | lte1 | 1.7 |
| ybr059c | akl1 | 2.7 |
| ybr125c | ptc4 | 1.1 |
| ybr279w | paf1 | 10.4 |
| ybr289w | snf5 | 11 |
| ydr264c | akr1 | 5.1 |
| ydr332w | | 9 |
| yer116c | slx8 | 5 |
| ygl115w | | 1.6 |
| yhl025w | snf6 | 3 |
| yjl138c | tif2 | 6.1 |
| yjl176c | swi3 | 2.6 |
| yjr063w | rpa12 | 7 |
| ylr006c | ssk1 | 6.5 |
| ylr357w | rsc2 | 2.2 |
| yol004w | sin3 | 6.6 |

TABLE 7
Transporter related mutations - ratio of glutathione secretion
by mutant to amount secreted by parental strain

| | |
|---|---|
| | Ratio GSH secreted |
| transporters | mutant:parental |

| | | |
|---|---|---|
| yer056c | fcy2 | 6.9 |
| yjl212c | opt1/hgt1 | 3.3 |
| yor088w | yvc1 | 2.7 |
| ypl058c | pdr12 | 6.8 |
| ygl167c | pmr1 | 8 |

TABLE 8
Membrane potential related mutations - ratio of glutathione
secretion by mutant to amount secreted by parental strain

| | |
|---|---|
| | Ratio GSH secreted |
| Membrane potential | mutant:parental |

| | | |
|---|---|---|
| ydr276c | pmp3 | 5.5 |
| yjr059w | ptk2 | 11 |
| yll010c | psr1 | 1.6 |

TABLE 9
Protein sorting related mutations - ratio of glutathione
secretion by mutant to amount secreted by parental strain

| Protein sorting/ | | Ratio GSH secreted |
|---|---|---|
| Protein folding | | mutant:parental |
| yal002w | vps8 | 7.3 |
| ybr105c | vid24 | 2.3 |
| ybr131w | ccz1 | 10.5 |
| ycl008c | vps23 | 27 |
| | (stp22) | |
| ydl077c | vps39/vam6 | 5.3 |
| ydr027c | luv1/vps54 | 15.2 |
| ydr323c | pep7/vps19 | 28.1 |
| ydr456w | nhx1 | 5.8 |
| ydr484w | vps52/sac2 | 10.3 |
| ydr486c | vps60 | 19 |
| ydr495c | vps3 | 15.5 |
| ydr518w | eug1 | 12 |
| yel036c | anp1 | 4.8 |
| yel051w | vma8 | 6 |
| yer122c | glo3 | 6 |
| yfr019w | fab1 | 10.7 |
| ygl167c | pmr1 | 7.8 |
| ygl212w | vps43/vam7 | 5.2 |
| ygr284c | erv29 | 3.9 |
| yhl031c | gos1 | 22 |
| yhr012w | vps29 | 8.2 |
| yhr171w | apg7 | 3.9 |
| yjl029c | vps53 | 13.4 |
| yjl053w | vps26/pep8 | 6.9 |
| yjl154c | vps35 | 18.8 |
| yjr075w | hoc1 | 3.6 |
| yjr102c | vps25 | 29.5 |
| ykl002w | did4 | 37 |
| ykl041w | vps24 | 25.7 |
| ykl212w | sac1 | 3.2 |
| ykr001c | vps1 | 7.9 |
| ykr035w-a | did2 | 21 |
| yll040c | vps13 | 3.1 |
| ylr025w | vps32/snf7 | 23 |
| ylr1148w | vac1/pep3/ | 21 |
| | vps18 | |
| ylr119w | srn2/vps37 | 25 |
| ylr262c | ypt6 | 2.5 |
| ylr268w | sec22 | 6.6 |
| ylr360w | vps38 | 5.9 |
| ylr373c | vid22 | 3.7 |
| ylr396c | vps33 | 14 |
| ylr417w | vps36 | 26.2 |
| yml001w | ypt7 | 13.1 |
| yml071c | dor1 | 4.6 |
| yml097c | vps9 | 1.7 |
| ymr004w | mvp1 | 2.9 |
| ymr077c | vps20 | 28.9 |
| ynr006w | vps27 | 33.7 |
| yol018c | tlg2 | 1.4 |
| yor036w | pep12/vps6 | 21.2 |
| yor069w | vps5 | 7.5 |
| yor070c | gyp1 | 3.5 |
| yor089c | vps21 | 8.7 |
| yor106w | vam3 | 6.1 |
| yor132w | vps17 | 2.6 |
| ypl065w | vps28 | 29.4 |
| ypl120w | vps30 | 3.8 |
| ypl149w | apg5 | 2.7 |
| ybr127c | vma2 | 5.8 |
| yor332w | vma4 | 6 |
| ylr447c | vma6 | 6 |
| ypr036w | vma13 | 2.4 |
| ygr105w | vma21 | 6 |
| ypr173c | vps4 | 12 |
| yfl031w | hac1 | 8 |

TABLE 10
Miscellaneous mutations - ratio of glutathione secretion
by mutant to amount secreted by parental strain

| | |
|---|---|
| | Ratio GSH secreted |
| Miscellaneous | mutant:parental |

| | | |
|---|---|---|
| yal047c | spc72 | 5 |
| ybl007c | sla1 | 9.1 |
| ybr041w | fat1 | 1.8 |
| ycl007c | cwh36 | 7 |
| ygl025c | pgd1 | 1.4 |
| ygl127c | soh1 | 2.9 |
| ygr217w | cch1 | 2.2 |
| yhr185c | pfs1 | 2.1 |
| yil053w | rhr2 | 7 |
| yjl042w | mhp1 | 10.4 |
| yjl183w | mnn11 | 2.9 |
| ylr226w | bur2 | 5.9 |
| yml048w | gsf2 | 1.6 |
| ynl225c | cnm1 | 2.4 |
| ynl280c | erg24 | 3.9 |
| yol001w | pho80 | 3.9 |
| yor043w | whi2 | 3.9 |
| ypl022w | rad1 | 24 |
| ypl037c | egd1 | 11.4 |

### Example 2

### Glutathione Production (Extracellular and Intracellular) Relative to Growth Phase

The strain designated as BSO4 was grown as per Example 1, but intracellular and extracellular glutathione levels were determined at a number of timed intervals after inoculation into fresh medium. The parental strain was also grown and sampled in the same way.

The results are illustrated in FIG. 2.

A mutant having a deletion in the VPS27 gene was grown as per Example 1, but intracellular and extracellular glutathione levels were determined at 15, 17, 19, 21, 23, 25, 27, 29, 32, 36 and 44 hours after inoculation into fresh medium. The parental strain was also grown and sampled in the same way.

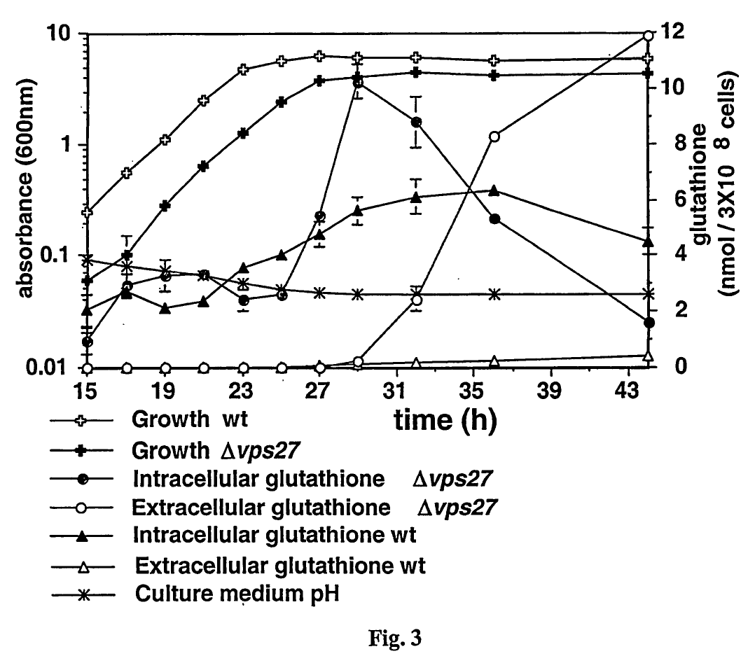

The results are illustrated in FIG. 3.

### Example 3

### Glutathione Production (Extracellular and Intracellular) Relative to pH

Several of the above mentioned strains from the BY4743 series were grown as follows.

Growth medium: As per SD minimal medium as described in Example 1, except the pH of the growth medium was buffered using a 25 mM PIPPS/MES buffer system (PIPPS=piperazine-N,N′-bis(2-ethanesulfonic acid) MES=2(N-morpholino)ethane sulfonic acid). The pH of the medium was adjusted to either pH 3.5 or pH 6.0 via the addition of ammonium hydroxide, or even a range of pH values were tested for strain BSO4.

Growth conditions and quantification of glutathione: The method used was identical to that outlined for the screening of the BY4743 series of deletion mutants.

The results (illustrated in FIGS. 7 to 9) show that extracellular GSH accumulation is greater if the pH of the growth medium is buffered at pH 3.5 vs pH 6.0. The differences observed in extracellular glutathione were determined to not be due to pH dependent degradation of glutathione.
Buffering the pH of the growth medium to pH 3.5 was found to increase the accumulation of extracellular glutathione in stationary phase cultures (48h) when compared to an equivalent culture grown in medium buffered to pH 6.0. To the best of our knowledge the effect of pH on the accumulation of extracellular GSH has not been reported.

Subsequent tests, using a broader selection of strains tested in either unbuffered SD medium or in SD medium buffered at pH6.0, identified the fact that different mutations are influenced in their glutathione secretion to different degrees by extracellular pH, although greater glutathione secretion was, except for in one instance, greater in the unbuffered medium. The results are summarised in Table 11 (glutathione levels provided as μM).

TABLE 11

| | | Total | | Total | |
|---|---|---|---|---|---|
| | | glutathione | | glutathione | |
| | Gene | SD medium | | SD medium | |
| Locus | name | (unbuffered) | S.D. | pH 6.0 | S.D. |
| BY4743 | Parent | 4.6 | 0.4 | 0.5 | 0.2 |
| yjl153c | ino1 | 40 | 3 | 27 | 3 |
| yol108c | ino4 | 15 | 3 | 22 | 2 |
| ypl002c | vps22 | 50 | 1 | 22 | 4 |
| ygl167c | pmr1 | 30 | 4 | 15 | 4 |
| ylr1148w | pep3 | 50 | 1 | 14 | 2 |
| ylr226w | bur2 | 14 | 3 | 12 | 2 |
| ylr114c | efr4 | 44 | 4 | 12 | 3 |
| ybr279w | paf1 | 25 | 1 | 9 | 2 |
| ybr289w | snf5 | 19 | 1 | 8 | 1 |
| ylr322w | | 20 | 2 | 6 | 1 |
| ylr396c | vps33 | 24 | 5 | 5 | 0.3 |
| yhl025w | snf6 | 13 | 7 | 5 | 0.1 |
| ygl127c | soh1 | 10 | 7 | 4 | 1 |
| ydr323c | pep7 | 31 | 7 | 4 | 1 |
| ydr495c | vps3 | 23 | 7 | 4 | 1 |
| ylr025w | vps32 | 40 | 7 | 4 | 1 |
| ycr063w | bud31 | 11 | 1 | 4 | 1 |
| ynl215w | | 7 | 1 | 3.4 | 0.3 |
| ymr077c | vps20 | 35 | 5 | 3.5 | 0.5 |
| yor036w | pep12 | 35 | 2 | 3.0 | 0.4 |
| ylr261c | | 18 | 3 | 3 | 1 |

These results also suggest that the combination of mutations where glutathione production is highly dependent on external pH, with those that are less-dependent on external pH could produce cells that produce even higher levels of glutathione.

### Example 4

### Glutathione Production (Extracellular and Intracellular) in the Presence of Different Monovalent Cations

To test the effect of various alkali metals on the relative secretion by deletion mutants, Saccharomyces cerevisiae laboratory strains designated CY4 and BSO4 were grown as follows.

Growth medium: As per SD minimal medium except the pH of the growth medium was buffered to pH 3.5 using a 25 mM PIPPS/MES buffer system. The growth medium contained either 150 mM KCl, RbCl or CsCl. The pH of the medium was adjusted to pH 3.5 via the addition of conc. ammonium hydroxide. The effect of adding combinations of the above salts was not studied.

The effect of the addition of theses salts was also confirmed in unbuffered medium.

Growth conditions and quantification of glutathione: the method used was identical to that outlined for the screening of the BY4743 series of deletion mutants.

Results: The addition of some alkali metal salts was shown to increase the accumulation of extracellular glutathione
The addition of the following salts at 150 mM concentration in the growth medium was also correlated with the increase accumulation of extracellular glutathione (CY4 strain need to reference): KCl, RbCl, CsCl.

The results for strain BSO4 are illustrated in FIG. 10.

### Example 5

### Glutathione Production (Extracellular) in the Presence of Leucine, Isoleucine and Valine

In this experiment the extracellular glutathione production was determined for one mutant yol081w (ira2), which for the mass screen work carried a marker for leucine auxotrophy. That is the strain contained a mutation in the leucine biosynthetic pathway (LEU2 gene mutation) and therefore for all our experiments the medium outlined in example 1 was used for the screen, in particular containing
Leucine 0.131 g/L
isoleucine 0.066 g/L and
valine 0.059 g/L

The ira2 mutant was altered to carry the plasmid (vector) Yep13LEU2 to allow the strain to make its own leucine. The data (below) shows glutathione secreted by the ira2 mutant grown with the additional supplements vs the ira2Yep 13 transformant grown under identical conditions but without the supplements.

Glutathione in the medium was determined after the cultures reached stationary phase using the conditions identical to those outlined in Example 1 (glutathione results provided as μmole/L).
GSH expressed as nanomoles of GSH per 3×10⁷ cells
Parental strain without the Yep13 plasmid=0.43
ira2=10.6
ira2Yep13=0.98

This data shows that if the strain can make leucine and leucine content of the culture is not regulated, then the strain secretes less GSH (therefore manipulating leucine in the medium for a strain that can make leucine is likely to have little effect—strains that can make normal levels of leucine are likely to secrete lower levels of GSH).

The extracellular GSH following growth in standard medium containing the above mentioned levels of leucine, isoleucine and valine (1×) was also compared to production in medium containing 2× leucine/isoleucine/valine (ie leucine 0.262 g/L, isoleucine 0.132 g/L and valine 0.118 g/L), and to 4× leucine/isoleucine/valine (leucine 0.524 g/L, isoleucine 0.264 g/L and valine 0.236 g/L).

Three strains were tested, 2 at all three concentrations of supplements, and 1 at two concentrations.

At 1× supplements: extracellular GSH per 3×10⁷ cells.
yor360c(pde2) produced 5.4±0.4
ynr006w (vps27) produced 12.3±0.5
ykl002w (did4) produced 14.0±0.8

At 2× supplements: extracellular GSH per 3×10⁷ cells.
yor360c (pde2) produced 0.47±0.07
ynr006w (vps27) produced 3.04±0.3
ykl002w (did4) produced 2.4±0.3

At 4× supplements: extracellular GSH per 3×10⁷ cells.
yor360c (pde2) produced 0.43±0.03
ynr006w (vps27) produced 2.0±0.1
ykl002w (did4) Not tested

The data show that supplementation of the leucine mutants with 2× leucine/isoleucine/valine resulted in a dramatic reduction in GSH secretion by these mutants.

Further experiments, using the same growth conditions and media described above, but with a broader range of strains, have indicated that different mutations are influenced in their glutathione secretion to different degrees by branched chain amino acid levels.

Table 12 provides data for strains which were strongly responsive to branched chain amino acid levels in the culture medium, and Table 13 provides data for strains which were less responsive to branched chain amino acid levels in the culture medium.

TABLE 12
Deletants secreting lower levels of glutathione following growth in
medium supplemented with increased branched-chain amino acids (BCAA)

| | | Glutathione^{a} | | Glutathione^{a} | | Glutathione^{a} | |
|---|---|---|---|---|---|---|---|
| Locus | Gene name | 1× BCAA | S.D. | 2× BCAA | S.D. | 4× BCAA | S.D. |
| BY4743 | parent | 5 | 1 | 5 | 0 | 4 | 2 |
| ynl229c | ure2 | 26 | 7 | 2.8 | 0.5 | 1.9 | 0.5 |
| yhl023c | | 33 | 3 | 20 | 4 | 4.2 | 0.3 |
| yol138c | | 34 | 1 | 16 | 1 | 4.8 | 0.4 |
| yel062w | npr2 | 35 | 2 | 22 | 5 | 5 | 2 |
| yol027c | | 30 | 1 | 12 | 1 | 5 | 1 |
| ylr119w | vps37 | 38 | 8 | 10 | 1 | 7 | 1 |
| yol050c | | 16 | 1 | 12 | 1 | 3 | 0 |
| yjl056c | zap1 | 25 | 7 | 15 | 7 | 5 | 1 |
| ybr003w | cox1 | 33 | 0 | 23 | 3 | 6 | 2 |
| ynr005c | | 38 | 11 | 12 | 1 | 8 | 1 |
| ycl008c | vps23 | 38 | 10 | 14 | 1 | 8 | 2 |
| yjr102c | vps25 | 40 | 8 | 16 | 0 | 9 | 1 |
| yor375c | gdh1 | 12 | 5 | 4 | 0 | 3 | 1 |
| yol004w | sin3 | 37 | 6 | 11 | 1 | 9 | 1 |
| ydr486c | vps60 | 37 | 2 | 28 | 3 | 9 | 7 |
| ydr276c | pmp3 | 29 | 6 | 16 | 5 | 7 | 1 |
| yjl188c | bud19 | 32 | 6 | 18 | 0 | 8 | 1 |
| ylr417w | vps36 | 36 | 7 | 15 | 1 | 10 | 1 |
| ykl002w | vps2 | 52 | 0 | 28 | 3 | 14 | 1 |
| ykr035w-a | did2 | 24 | 9 | 14 | 2 | 7 | 2 |
| ypr004c | | 34 | 2 | 15 | 1 | 9 | 1 |
| ylr025w | vps32 | 40 | 7 | 19 | 1 | 12 | 2 |
| yfr010w | ubp6 | 20 | 2 | 11 | 2 | 6 | 1 |
| ykl213c | doa1 | 24 | 5 | 16 | 1 | 7.6 | 0.1 |

^{a}Extracellular glutathione concentration in μm. 24 most-responsive deletants shown (based on [GSH secretion in SD medium]/] GSH secretion in SD containing 4× BCAA supplements])

TABLE 13
Glutathione oversecreting deletants less responsive to increased
branched-chain amino acid (BCAA) supplementation

| | | Glutathione^{a} | | Glutathione^{a} | | Glutathione^{a} | |
|---|---|---|---|---|---|---|---|
| Locus | Gene name | 1× BCAA | S.D. | 2× BCAA | S.D. | 4× BCAA | S.D. |
| BY4743 | parent | 5 | 1 | 5 | 0 | 4 | 2 |
| ylr1148w | pep3 | 50 | 1 | 48 | 2 | 50 | 2 |
| ylr396c | vps33 | 24 | 6 | 30 | 6 | 41 | 6 |
| yor036w | pep12 | 35 | 2 | 38 | 5 | 28 | 1 |
| ydr323c | pep7 | 31 | 1 | 35 | 5 | 26 | 5 |
| ydr027c | luv1 | 32 | 2 | 31 | 1 | 35 | 3 |
| ydr484w | sac2 | 35 | 4 | 32 | 3 | 27 | 1 |
| yfr019w | fab1 | 28 | 0 | 28 | 0 | 26 | 4 |
| ykr001c | vps13 | 23 | 3 | 22 | 1 | 25 | 1 |
| ydr495c | vps3 | 23 | 1 | 28 | 0 | 18 | 2 |
| ynl297c | mon2 | 23 | 4 | 20 | 6 | 18 | 2 |
| yor070c | gyp1 | 18 | 2 | 19 | 1 | 17 | 0 |
| yjl102w | mef2 | 24 | 2 | 22 | 1 | 24 | 3 |
| yol081w | ira2 | 18 | 3 | 18 | 1 | 17 | 3 |
| yjl153c | ino1 | 40 | 3 | 37 | 1 | 39 | 6 |
| yol108c | ino4 | 14 | 3 | 15 | 2 | 16 | 1 |
| ylr114c | efr4 | 44 | 4 | 35 | 2 | 36 | 1 |
| yjl095w | bck1 | 13 | 2 | 15 | 1 | 18 | 1 |
| yhr030c | mpk1 | 12 | 2 | 12 | 1 | 15 | 0 |
| ydr264c | akr1 | 18 | 1 | 12 | 4 | 19 | 4 |
| yjl042w | mhp1 | 29 | 1 | 26 | 1 | 25 | 3 |
| yal047c | spc72 | 14 | 1 | 14 | 2 | 12 | 2 |
| ycl007c | cwh36 | 14 | 2 | 12 | 2 | 11 | 1 |
| ydl074c | bre1 | 22 | 6 | 16 | 1 | 21 | 4 |
| yer116c | slx8 | 15 | 4 | 21 | 0 | 24 | 6 |
| ynr036c | | 23 | 2 | 24 | 3 | 21 | 1 |
| ybr056w | | 15 | 2 | 16 | 0 | 21 | 5 |
| yjl176c | swi3 | 10 | 2 | 11 | 1 | 14 | 1 |
| yil029c | | 33 | 1 | 26 | 3 | 25 | 1 |

^{a}Extracellular glutathione concentration in μm. 24 most-responsive deletants shown (based on [GSH secretion in SD medium]/] GSH secretion in SD containing 4× BCAA supplements])

These results also suggest that the combination of mutations where glutathione production is strongly responsive to branched chain amino acid levels, with those that are less-responsive to branched chain amino acid levels could produce cells that produce even higher levels of glutathione.

### Example 6

### Glutathione Production in the Presence of Myo-Inositol

Manipulation of myo-inositol content alone or together with additional glucose supplementation (or potentially other carbon sources such as respiratory carbon sources) in the culture medium was found to influence glutathione production.

A wild-type haploid laboratory strain, CY4, was grown in SD medium (either by itself (open bars, 2% glucose), or SD medium supplemented by 200 mg/L myo-inositol (hatched bars), 4% w/v glucose (final glucose concentration—shaded bars), or both 200 mg/L myo-inositol and 4% w/v glucose (solid bars)), and the culture medium assayed for external glutathione as described in Example 1. The data shown are means (±standard deviation) for triplicate measurements from a representative experiment.

The results, illustrated in FIG. 11, show the effect of increased myo-inositol supplementation, either alone or in combination with glucose, on glutathione production (extracellular levels). While glucose supplementation alone did not appear to affect the amount of extracellular glutathione produced, when glucose supplementation was combined with myo-inositol supplementation, significantly greater amounts of extracellular glutathione were produced relative to growth in SD medium, or SD medium supplemented with myo-inositol alone.

Thus, myo-inositol supplementation, optionally combined with elevated levels of carbon source/substrate can result in elevated extracellular glutathione production by yeast strains.

### Example 7

### Glutathione Production by Combined/Double Mutants

A number of yeast strains having mutations in two of the genes referred to in Table 1 above (and/or having two mutations as identified tables 2 to 10) have also been found to provide elevated extracellular glutathione production. A number of these double mutants provide greater extracellular glutathione production than strains having either mutation alone. FIGS. 12 to 14 provide examples of this (media and methods as described in Example 1).

FIG. 12 illustrates the extracellular glutathione levels produced by:
a wild-type (wt) yeast strain;
a mutant of the wild-type having the BSO4 mutation (defect in the HAC1 gene, YFL031 W, identified in a BY4742 strain background);
a mutant of the wild-type having the ycf1 (ydr135c) mutation; and
a yeast strain having combined BSO4 and ycf1 mutations as HAC1 then the bso4 ycf1 double mutant can be listed as hac1 ycf1 (in place of bso4 ycf1).

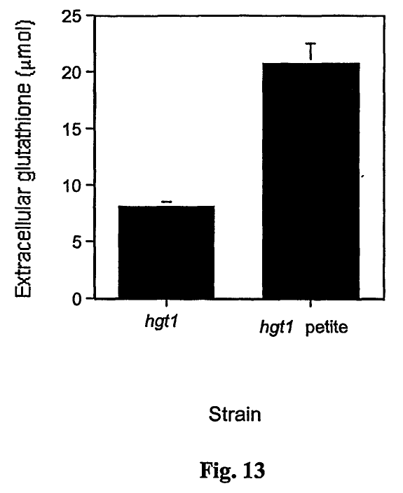

FIG. 13 illustrates the extracellular glutathione levels produced by a yeast strain having the hgt1 mutation (glutathione uptake (re-uptake) mutation), and a yeast strain having combined hgt1 and petite (mitochondrial respiratory deficiency) mutations. The data shown are means (±S.D.) for triplicate measurements from a representative experiment.

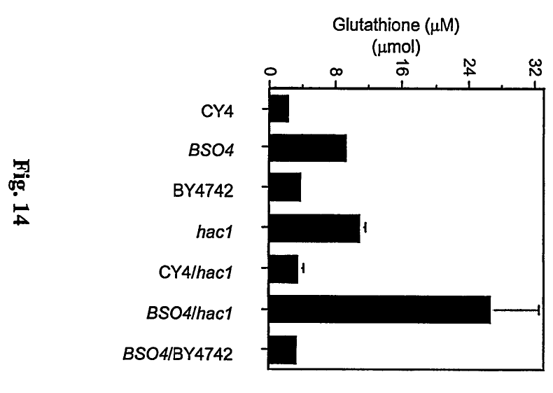

FIG. 14 illustrates the extracellular glutathione levels produced by different haploid wild-types (CY4 and BY4742), single mutants thereof, and different diploid crosses. The diploid strain generated by mating a bso4 haploid to a hac1 deletant (hence a double mutant) produces diploid cells that produce higher levels of glutathione relative to either of the respective haploid strains or diploids derived from mutant-wild-type crosses (single mutant diploids. The hac1 deletant is derived from the BY4742 the strain background and BSO4, which carries a mutation in HAC1, is derived from the CY4 strain background). The data shown are means (±S.D.) for triplicate measurements from a representative experiment.

1. A process for the production of glutathione, wherein said process comprises culturing a mutant yeast strain under conditions promoting glutathione production, and wherein said yeast strain has one or more genetic mutations that result in increased secretion of glutathione into the culture medium relative to a parental strain and optionally isolating the glutathione from the culture medium.

2. The process of claim 1, wherein the yeast strain has a mutation that reduces the ability of the strain to synthesize one or more proteins, metabolites or essential growth factors, and wherein said metabolites or essential growth factors are included in the culture medium in limiting amounts.

3. The process of claim 2, wherein said metabolites or essential growth factors are selected from amino acids, or precursors or metabolites thereof.

4. The process of claim 3, wherein the yeast is deficient, or has a reduced ability for synthesis of, leucine, isoleucine or valine, or a combination thereof, or precursors or metabolites thereof.

5. The process of claim 1, wherein the yeast strain has at least one mutation selected from the group consisting of:
i) mutation in a gene or genes encoding components of the mitochondrial respiratory chain or nuclear genes encoding proteins that maintain the integrity of the mitochondrial genome or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26S proteosome;
viii) mutation in a gene or genes involved in transportation of glutathione across the yeast cell membrane;
ix) mutation in a gene or genes involved in glutathione degradation; and
x) mutation in a gene or genes involved in vacuolar function.

6. The process of claim 5, wherein said yeast strain also has a mutation that reduces the ability of the strain to synthesize one or more proteins, metabolites or essential growth factors, and wherein said metabolites or essential growth factors are included in the culture medium in limiting amounts.

7. The process of claim 6, wherein said metabolites or essential growth factors are selected from amino acids, or precursors or metabolites thereof.

8. The process of claim 7, wherein the yeast is deficient, or has a reduced ability for synthesis of, leucine, isoleucine or valine, or a combination thereof, or precursors or metabolites thereof.

9. The process of claim 1, wherein said yeast strain also overexpresses the glutathione synthesis pathway.

10. The process of claim 9, wherein said yeast strain overexpresses gammaglutamylcysteine synthetase (GSH1), glutathione synthetase (GSH2), or both.

11. The process of claim 1, wherein the yeast culture is grown aerobically.

12. The process of claim 1, wherein the yeast culture is grown at a pH of from about 2.5 to about 5.

13. The process of claim 1, wherein the yeast culture is grown in the presence of monovalent cations selected from the group consisting of sodium, potassium, rubidium, caesium, and combinations thereof.

14. The process of claim 13, wherein the concentration of said monovalent cations in the culture medium is from about 100 mM to about 250 mM.

15. The process of claim 1, wherein the yeast culture is grown in the presence of myo-inositol.

16. The process of claim 15, wherein the concentration of the myo-inositol in the culture medium is from about 0.1 mM to about 10 mM.

17. The process of claim 15, wherein the yeast is also grown in the presence of elevated levels of a carbon source selected from the group consisting of fermentable sugars, non-fermentable carbon sources, oligosaccharides which are homo- or hetero-oligomers comprising fermentable sugar moieties, and combinations thereof.

18. The process of claim 17, wherein said carbon source is selected from the group consisting of ethanol glucose, fructose, sucrose, and combinations thereof.

19. The process of claim 17, wherein the concentration of said carbon source in the initial uninoculated culture medium is from about 2% to about 10% w/v.

20. The process of claim 19, wherein the concentration of said carbon source in the initial uninoculated culture medium is about 4% w/v.

21. The process of claim 1 which comprises dough preparation or preparation of a fermented product.

22. A mutant yeast strain having at least two mutations selected from the group consisting of:
i) mutation in a gene or genes encoding components of the mitochondrial respiratory chain or nuclear genes encoding proteins that maintain the integrity of the mitochondrial genome or mutation or deletion of the mitochondrial genome;
ii) mutation in a gene or genes affecting intracellular levels of NAD(P)H and NAD(P)⁺;
iii) mutation in a gene or genes affecting the assimilation and metabolism of nitrogen in the cell;
iv) mutation in a gene or genes encoding regulatory components of the Ras/cAMP/PKA pathway or otherwise affecting the activity of the Ras/cAMP/PKA pathway;
v) mutation in a gene or genes affecting endosomal function;
vi) mutation in a gene or genes affecting the Golgi to endosome to vacuole transportation pathway or plasma membrane to endosome to vacuole traffic;
vii) mutation in a gene or genes affecting ubiquitin levels and ubiquitin-mediated proteolysis via the 26S proteosome;
viii) mutation in a gene or genes involved in transportation of glutathione across the yeast cell membrane;
ix) mutation in a gene or genes involved in glutathione degradation; and
x) mutation in a gene or genes involved in vacuolar function.

23. The yeast strain of claim 22, which also has a mutation that reduces the ability of the strain to synthesise one or more proteins and wherein said metabolites or essential growth factors are included in the culture medium in limiting amounts.

24. The yeast strain of claim 23, wherein said metabolites or essential growth factors are selected from amino acids, or precursors or metabolites thereof.

25. The yeast strain of claim 24, which is deficient, or has a reduced ability for synthesis of, leucine, isoleucine or valine, or a combination thereof, or precursors or metabolites thereof.

26. The yeast strain of claim 22, which also overexpresses the glutathione synthesis pathway.

27. The yeast strain of claim 26, which overexpresses gammaglutamylcysteine synthetase (GSH1), glutathione synthetase (GSH2), or both.

28. A method of preparing a dough comprising combining a yeast strain of claim 22 with other dough components.

29. A method of producing a fermented product comprising adding to the unfermented precursor component(s) of said product a yeast strain of claim 22.
